(19) United States
(12) Patent Application Publication
(10) Pub. No.: US 2015/0004112 A1
(43) Pub. Date: Jan. 1, 2015

(54) SILOXANE POLYMERS WITH A CENTRAL POLYSILOXANE POLYMER BLOCK WITH ORGANOFUNCTIONAL RADICALS EACH HAVING AT LEAST TWO BIVALENT GROUPS SELECTED FROM UREA AND/OR CARBAMATE GROUPS AND AT LEAST ONE UV/VIS CHROMOPHORE AS RADICAL

(71) Applicant: EVONIK INDUSTRIES AG, Essen (DE)

(72) Inventors: Helmut Ritter, Wuppertal (DE); Wilfried Knott, Essen (DE); Frauke Henning, Essen (DE); Christian Hartung, Essen (DE); Berit Knudsen, Erkrath (DE)

(21) Appl. No.: 14/313,209

(22) Filed: Jun. 24, 2014

(30) Foreign Application Priority Data
Jul. 1, 2013 (DE) 102013106905.3

(51) Int. Cl. C08G 77/388; A61Q 5/12; A61Q 19/00; C11D 3/00; A61Q 5/02; A61Q 19/10; A61Q 5/00; A61K 8/898; A61Q 5/06

(52) U.S. Cl. CPC C08G 77/388; A61K 8/898; A61Q 5/12; A61Q 19/00; A61Q 5/065; A61Q 5/02; A61Q 5/06; A61Q 19/10; A61Q 5/006; C11D 3/001; A61K 2800/522
USPC 424/70.6; 525/474; 525/454; 525/459; 549/293; 424/70.12; 424/70.122; 424/78.02; 510/122; 510/130; 510/515

(57) ABSTRACT

The invention relates to siloxane polymers comprising a central polysiloxane polymer block B with organofunctional radicals bonded terminally or laterally to the polymer block and based on IPDI and UV/Vis chromophores or derivatives, in particular UV chromophores, which are covalently bonded via a hydrophobic or hydrophilic linker group Q1′, Q2′, and compositions comprising these siloxanes. Also disclosed are processes for their preparation and their use.

The present application claims priority from German Patent Application No. DE 10 2013 106 905.3 filed on Jul. 1, 2013, the disclosure of which is incorporated herein by reference in its entirety.

### FIELD OF THE INVENTION

The invention relates to siloxane polymers comprising a central polysiloxane polymer block B with organofunctional radicals that are terminal or bonded laterally to the polymer block, based on preferably IPDI and UV/Vis chromophores or derivatives thereof, in particular on UV chromophores which are covalently bonded via a hydrophobic or hydrophilic linker group Q1′, Q2′, and to compositions comprising these siloxane polymers. Also disclosed are processes for their preparation and their use.

For the treatment and modification of the properties of textile fibres and keratin fibres and also of the skin, the property profile and the attained effect in the area of haircare, body care or treatment of textiles can be considerably improved by adding modified siloxanes as additives. For example, essential product properties can be improved considerably by adding modified silicones. Mention is to be made of the improved suppleness of creams, the skin feel, the shine of hair or its combability, and also the water resistance of sun creams.

It is known that UV light of wavelengths from 200 to 380 nm or short-wave Vis light 320 to 400 nm is responsible for the bleaching and the damage of textiles, synthetic fibres and natural fibres (e.g. wool, cotton and hair).

In order to expand the application spectrum of the siloxanes, in particular nitrogen-containing siloxanes, there is a need for further modified siloxanes. A particular focus here is on siloxanes whose backbone deliberately has regions with different properties. Thus, there is a need for siloxanes which have hydrophobic regions and at the same time regions which are hydrophilic or water-soluble. There is a particular need for siloxanes which are able to add via hydrogen bridge bonds onto natural surfaces, such as keratin fibres or else textile natural fibres. The siloxanes should particularly preferably be adjustable as regards their hydrophilicity and/or their hydrophobicity. Preferably, the aim is to develop a siloxane or a mixture of siloxanes which can be used both as an additive in cosmetic formulations or in the treatment of textiles in order to shield the skin, keratin fibres, textiles, synthetic fibres and natural fibres against UV radiation or to permit control via the degree of damage. There is therefore a growing need for UV radiation-absorbing substances. It is therefore desirable to provide compounds which are able to exert control over the UV radiation to which the textiles or the synthetic or natural fibres are subjected.

A large number of compounds are known from the literature which are used for the UV light protection of fibres, dyes and pigments. Such compounds are typically used directly in the manufacture of the fibres.

However, these compounds do not display a care or softening effect. Additionally, they often lack adhesion to the fibre surface, meaning that the UV protection on the fibres is lost after just a few washing operations.

Moreover, it is desirable to provide compounds which exert a care effect on natural or synthetic fibres, exhibit a high substantivity on the fibres and additionally offer protection against damage as a result of mechanical and/or optical (e.g. UV light) influences. In order to be able to provide adequate UV protection even after washing several times, the compounds should also be able to be incorporated into fabric softener formulations and attach to the fibres during the softening operation.

It is noted that citation or identification of any document in this application is not an admission that such document is available as prior art to the present invention.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the EPO (Article 83 of the EPC), such that applicant(s) reserve the right to disclaim, and hereby disclose a disclaimer of, any previously described product, method of making the product, or process of using the product.

### OBJECTS OF THE INVENTION

The complex object of the invention consisted in providing modified siloxanes which can reduce UV damage of the surfaces treated with the siloxanes, such as fibres or textiles, and in which the content of UV-radiation-absorbing groups and also of nitrogen-containing groups, in particular of tertiary or quaternary nitrogen-containing groups and UV-radiation-absorbing groups can be varied arbitrarily independently of one another. Moreover, the substantivity of these compounds for natural or synthetic fibres (e.g. wool, cotton or hair) or other surfaces (e.g. the skin) should be able to be adjusted as desired. By virtue of the siloxane chain, the aim is to achieve a smooth, care and softening effect on natural or synthetic fibres. A further aim consisted in providing a UV-radiation-absorbing Gemini surfactant (bis-surfactant or double-surfactant) which is suitable for use in the cosmetics sector, in the textile industry, in detergent formulations, and as additive for influencing the surface properties of paints, moulding compositions etc., and can for example influence the spreading of water drops etc. in a positive manner.

The objects are achieved by the siloxane according to the invention and compositions comprising these corresponding to the features of Claims 1 and 24 and 25 relating to the composition according to the invention comprising at least one siloxane according to the invention, and also by the process for the preparation according to Claim 17 and also the formulation according to the invention corresponding to the features of patent Claim 27.

Surprisingly, the objects were achieved by providing siloxane polymers comprising a central polysiloxane polymer block B and at least two terminal or at least one terminal and at least one lateral organofunctional group, which are each derived from a reaction of an isocyanate group from diisocyanates with a UV/Vis chromophore, preferably with at least one absorption maximum in the range from 300 to 380 nm optionally with a second absorption maximum in the range from 250 to 299 nm, preferably a UV chromophore, which preferably has at least one absorption maximum in the range from 280 to 380 nm, which preferably has at least two absorption maxima in this range. As UV chromophore, particular preference is given to using coumarin or a coumarin derivative, such as a hydroxy- or amino-functional coumarin, coumarin derivative or salt thereof, where the formed UV chromophore isocyanate, in particular coumarin isocyanate is bonded to the polysiloxane via a linker (Q1′, Q1′AH, Q2′, Q2′AH). Suitable linkers are preferably compounds containing alkylene, —(CH₂)ₙ (n=2 to 200), aryl, arylalkylene, optionally with heterotatoms O, N and/or S, aminoalkylene, quaternary aminoalkylene, allyl, ester, amide, anhydride, urea, (meth)acrylate groups, and substituted and unsubstituted polyethers, in particular polyethylene glycol [EO]v, propylene glycol [PO]w or [EO]v[PO]w.

### SUMMARY OF THE INVENTION

The invention provides at least one siloxane polymer of the general formula I comprising a central polysiloxane polymer block B,

(i.) which is substituted with organofunctional radicals which comprise organofunctional radicals, preferably an alkylene radical with 1 to 22 carbon atoms and/or a phenyl group or a polyether,

(ii) the polymer block B has linear and/or branched structures with at least two difunctional siloxane units,

(iii.) the polymer block B has on at least two terminal silicon atoms or at least one terminal and at least one lateral silicon atom of the siloxane units of polymer block B the organofunctional radicals -Q1 and -Q2, where the radicals are identical or different,

Q2-B-Q1 (I)

where -Q1 corresponds to the general formula IIa and -Q2 corresponds to the formula IIb, which are independently dentical or different,

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)
where A is —NH—, —O— or —S— and D is —NH— in each case independently in formulae IIa and IIb,
where A′ is —NH— and D′ is —NH—, —O— or —S—, preferably —O— in each case independently
in formulae IIa and IIb, where each radical Q1 and Q2 of the formulae IIa or IIb
has at least two bivalent groups, selected from carbamate and urea
group, preferably IIa and IIb can in each case have two carbamate groups or
one urea and one carbamate group or two urea groups,
with Q1′ and Q2′ in each case independently comprising a bivalent hydrocarbon radical with 6 to 200 carbon atoms optionally comprising at least one
heteroatom O, N or S, a bivalent radical comprising aryl, arylalkyl groups
optionally comprising at least one heteroatom O, N or S or polyether radicals containing alkyl, aryl or
alkyl and aryl groups,
with Q1″ and Q2″ in each case independently comprising a bivalent linear, branched and/or cyclic alkyl radical with 4 to 200 carbon atoms, in particular cyclohexenyl radical based, as from the reaction of IPDI or a bivalent
radical comprising an aryl and/or arylalkyl radical with 6 to 200 carbon atoms, and
with -D′-Q1* and -D′-Q2* with D′, as defined above, where -D′-Q1* and -D′-Q2* in each case independently comprise as radicals Q1* and Q2* a UV/Vis chromophore as radical, which preferably has at least one absorption maximum between 300 and 380 nm, particularly preferably the chromophore has at least two absorption maxima between 280 and 340, which are preferably between 280 and 299 nm and between 300 and 340 nm. Preferably, D′ in the two radicals -D′-Q1* and -D′-Q2* where D′=—O—. The carbamate groups are both —O—(C═O)—NH— and —S—(C═O)—NH— groups, which are also referred to as thiocarbamates (thiolurethane).

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

Chromophores preferred according to the invention are UV chromophores, preferably UVA2 chromophores, which preferably have at least one absorption maximum in the range from 320 to 340 nm and/or a UVB chromophore, which has in particular an absorption maximum in the range from 280-320 nm, particularly preferably the UVB chromophore has two absorption maxima, such as for example between 280 and 299 nm and between 300 and 320 nm. According to the invention, preference is given to coumarin, coumarin derivatives or salts thereof.

Preferably, B corresponds to formulae IIIa or IIIb. Preferably, Q1′ or Q2′ is in each case independently a bifunctional linear, branched or cyclic alkylene radical with 1 to 22 carbon atoms, a bifunctional aryl, arylalkylene radical with 6 to 30 carbon atoms or polyether. And preferably with Q1″ and Q2″, which are in each case independently bifunctional linear, branched or cyclic alkylene group with 1 to 22 carbon atoms or bifunctional aryl, arylalkylene radicals with 6 to 30 carbon atoms.

In the strict sense, however, the term chromophore refers only to the part of a substance which is responsible for imparting the colour (colour carrier).

A UV chromophore shows a good absorption behaviour in the spectral range of the UV rays or preferably an absorption maximum. Here, the chromophore absorbs the energy of the ultraviolet light and preferably does not change chemically as a result. The energy is released as heat or phosphorescence/fluorescence. Alternatively or additionally, the chromophore can bring about molecular changes to itself or in its environment. The chromophores according to the invention are preferably characterized in that they release the energy as heat or phosphorescence/fluorescence. The best known UV chromophore is melanin. An important UV chromophore is also DNA itself. Its absorption maximum is at 260 nm. A UV chromophore according to the invention has at least one absorption maximum between 280 and 380 nm, preferably it has at least two absorption maxima in this range, preferably in the range from 280 to 340, particularly preferably at least one maximum is in the range from 315 to 340 nm. One suitable UVA chromophore according to the invention is a compound with at least one absorption maximum between 315 and 380 nm, a preferred UVA chromophore according to the invention is a UVA2 chromophore with at least one absorption maximum in the range from 315 to 340 nm. Suitable UVB chromophores according to the invention are compounds with at least one absorption maximum in the range from 280 to 315 nm, preferred UVB chromophores have at least two absorption maxima in this range. The Vis chromophores include compounds with absorption maxima from 320 to 790, where the VIS chromophores according to the invention for a pure UV protection include only the colourless Vis chromophores with an absorption maximum from 380 to 400 nm, while according to an alternative b) also coloured Vis chromophores with absorption maxima from 400 to 790 nm can be used if an intrinsic colour is desired. As pure UV protection, only colourless siloxane polymers can be used so as not to change the coloured appearance of a treated surface.

According to a particularly preferred embodiment, the siloxane polymers according to the invention of the general formulae I, IIa and IIb have as -D′-Q1* and -D′-Q2* in each case independently as radicals a UV chromophore comprise with at least one absorption maximum in the range from 280 to 380 nm, particularly preferably with at least one absorption maximum from 300 to 380 nm, they preferably comprise a UVA2 chromophore as radical with at least one absorption maximum from 315 to 340 nm, preferably from 320 to 340 nm and/or a UVB chromophore with at least one absorption maximum in the range from 280-315 nm, preferably with at least one absorption maximum in the range from 300 to 315 nm.

According to one embodiment, the siloxane polymers of the general formulae I, IIa and IIb in -D′-Q1* and -D′-Q2* comprise in each case independently a Vis chromophore as radical with at least one absorption maximum in the range from 320 to 790 nm, in particular 320 to 380 nm and/or a Vis chromophore, which absorbs between 420 and 790 nm.

Siloxanes with the following substitution pattern have particularly advantageous properties as regards improved UV protection of keratin fibres, in particular hair. Consequently, the invention further provides at least one siloxane polymer or a composition comprising at least one corresponding siloxane polymer or a mixture of these, comprising the radicals -Q1 and -Q2 of the formula I where A is —O—, D is —NH—, A′ is —NH— and D′ is —NH— or —O—, in particular where D′ is —O—, where the radicals -D′-Q1* and -D′-Q2* are in each case independently derived from hydroxy coumarin, hydroxy coumarin derivatives or amino-functional coumarin derivatives.

According to one embodiment, the radicals -Q1 and -Q2 of the formula I comprise the radicals -D′-Q1* and -D′-Q2*, which are in each cased derived independently from a hydroxycoumarin or hydroxycoumarin derivative, particularly preferably from 7-hydroxycoumarin (CAS: 93-35-6), 4-hydroxycoumarin (CAS: 1076-38-6), 6-hydroxycoumarin (CAS: 6093-68-1) or an amino-coumarin, amino-coumarin derivative, such as 7-amino-4-methylcoumarin (coumarin 120), hydroxy-isocoumarin.

Further preferably, the radicals comprise -Q1 and -Q2 of the formula I for A is —O—, D is —NH—, A′ is —NH— and D′ is —O—, and the radicals -D′-Q1* and -D′-Q2* are in each case independently derived from a hydroxycoumarin or hydroxycoumarin derivative, in particular 7-hydroxycoumarin, 4-hydroxycoumarin or 6-hydroxycoumarin.

According to one alternative, the siloxane polymer of the general formulae I, IIa and IIb comprises radicals, in particular additional radicals which are selected from -D′-Q1* and -D′-Q2* where D′ is —NH— with Q1* or Q2* in each case independently an amino-functional hydrocarbon radical, in particular with a tertiary or quaternary nitrogen atom. These radicals -D′-Q2* and -D′-Q1* are provided according to the invention in addition to the —O-Q2* and —O-Q1* radicals comprising coumarin in a siloxane of the formula I. The nitrogen-containing radicals bring about improved substantivity on keratin fibres.

In all general formulae it is the case that the following symbol [symbol] indicates a bonding site/monovalent bonding site, onto which is covalently bonded—not shown—an atom, a group or radical. In the case of substituted cyclohexyl radicals based on the diisocyanate isophorone radicals, the NH(C═O)A- or NH(C═O)D′- radicals are bonded to the bonding sites [symbol] . In the formulae IIIa, IIIb, the radicals Q2- and -Q1 are covalently bonded to [symbol] .

According to a particularly preferred embodiment, the invention provides at least one siloxane polymer of the general formula I or mixtures of these, in which the polymer block B corresponds to at least one of the general formulae IIIa or IIIb, where B is

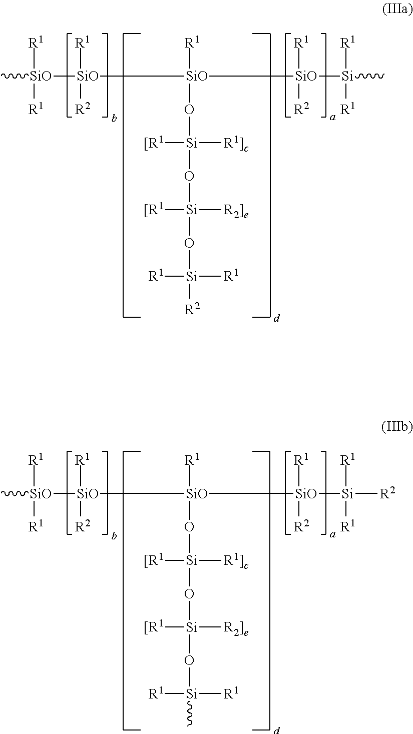

where a, b, c, d and e in formulae IIIa and IIIb are in each case independently an integer

where a is from 1 to 200, in particular 2 to 150, preferably 2 to 100, particularly preferably 5 to 100, preferably 20 to 100,

where b is from 0 to 200, in particular 2 to 150, preferably 2 to 100, particularly preferably 5 to 100, preferably 20 to 100,

where c is from 0 to 200, in particular 2 to 150, preferably 2 to 100, particularly preferably 5 to 50, preferably 5 to 20,

where d is from 0 to 200, in particular 2 to 150, preferably 2 to 100, particularly preferably 5 to 100, preferably 20 to 100,

where e is from 0 to 200, in particular 2 to 150, preferably 2 to 100, particularly preferably 5 to 50, preferably 5 to 20, where (a+b+c+d+e) is greater than or equal to 1, preferably greater than or equal to 20, and with R¹ in formula IIIa or IIIb in each case independently identical or different, where R¹ comprises alkyl radicals with 1 to 22 carbon atoms, preferably 1 to 4 carbon atoms, particularly preferably 1, 2, 3, 4, 6 carbon atoms or phenyl radicals, where R² in formula IIIa or IIIb is alkyl radical with 1 to 22 carbon atoms, preferably 1 to 4 carbon atoms, particularly preferably 1, 2, 3, 4, 6 carbon atoms, an alkyl radical with at least one heteroatom selected from N, O, S, such as an alkylamine, alkylcarboxylic acid, alkylcarboxamide, alkylcarboxylic anhydride, (meth)acrylate, phenyl radical or a radical -Q1′-A-(C═O)-D-Q1″-NH₂ and/or Q2′-A-(C═O)-D-Q2″-NH₂. R¹ and R² are particularly preferably selected from alkyl groups with 1, 2, 3 or 4 carbon atoms, in particular from methyl groups or at least one R² is an aminoalkyl group, in particular with a primary amino group or a quaternary alkylamine group.

In particularly preferred siloxane polymers of the formula I, IIIa, and/or IIIb, the indices b, c, d and e are 0 and a is 2 to 200, in particular 2 to 100, preferably 20 to 100, in particular a is 30 or 80 with a variation of plus/minus 5.

According to the invention, the radicals -Q1 and -Q2 in the general formula I are independently selected from

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)

a) where A is —O—, D is —NH—, A′ is —NH— and D′ is —O—,

b) where A is —O—, D is —NH—, A′ is —NH— and D′ is —NH—,

c) where A is —NH—, D is —NH—, A′ is —NH— and D′ is —NH—,

d) where A is —S—, D is —NH—, A′ is —NH— and D′ is —NH—,

e) where A is —NH—, D is —NH—, A′ is —NH— and D′ is —O— or

f) where A is —S—, D is —NH—, A′ is —NH— and D′ is —O—,

g) where A is —O—, D is —NH—, A′ is —NH— and D′ is —S—,

h) where A is —NH—, D is —NH—, A′ is —NH— and D′ is —S— or

i) where A is —S—, D is —NH—, A′ is —NH— and D′ is —S—

with a) where A is —O—, D is —NH—, A′ is —NH— and D′ is —O— being particularly preferred.

Particularly preferred diisocyanates and urethanes derived therefrom that have proven suitable in the radicals -Q1 and -Q2 of the formula I are the bivalent radicals -Q1″- and -Q2″-, which are selected independently from bivalent, linear, branched or cyclic alkylene radicals with 4 to 25 carbon atoms, in particular with 6, 7, 8, 9, 10, 11, 12, 13 or 14 carbon atoms, preferably hexylene (—CH₂)₆, heptylene, bivalent 2,4-toluolyl, diphenylmethane, polymeric diphenylmethane, 3,5,5-trimethyl-1-methylen-3-ethylenecyclohexane derived from the reaction of IPDI or 4,4′-dicyclohexylene. According to the invention, particular preference is given to using isophorone diisocyanates (IPID), which, on account of the structural isomerism, permit good control of the process, since an isocyanate group is more reactive and consequently the formation of high molecular weight polymers can be avoided. The process is therefore very readily reproducible and the siloxanes are obtainable with defined molecular weights.

Siloxane polymers according to the invention can preferably comprise in the radicals -Q1 and -Q2 of the formula (I) at least as one of the bivalent radicals -Q1″- and -Q2″- independently a bivalent cyclohexane containing radical, selected from the formulae Va and Vb

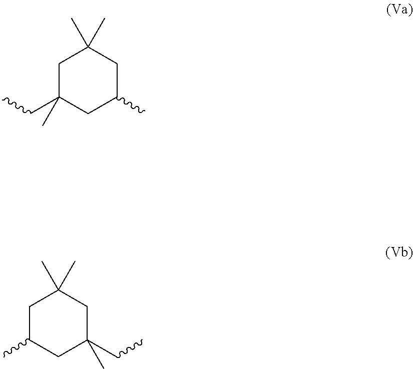

in particular, Q2″ is a bivalent cyclohexane containing radical of the formula Va and -Q1″ is of the formula Vb. Here, on account of the different reactivity of the isocyanate groups, the siloxane polymer, in particular of the formula I, preferably with the structure shown, in particular of the formulae Ia, Ib, Ia*, Ib*, is formed as main product by the process according to the invention by variant a), process variant b) preferentially leading to the formation of the siloxane polymers of the formula Ic.

The linker (-Q1′-A-, -Q2′-A-) is preferably derived from an olefinic alcohol with 3 to 200 carbon atoms, preferably with 3 to 25 carbon atoms, optionally with at least one heteroatom comprising N, O or S. Likewise preferred in the radicals -Q1 and -Q2 of the formula I are the bivalent radicals -Q1′- and -Q2′-, selected from alkylene radicals with 3 to 22 carbon atoms optionally with at least one heteroatom comprising N, O or S, in particular —(CH₂)ₙ— where n is from 3 to 22 optionally with at least one heteroatom comprising N, O or S, preference also being given to hexylene (—CH₂)₆—, heptylene (—CH₂)₇—, octylene (—CH₂)₈—, nonylene (—CH₂)₉—, decylene (—CH₂)₁₀—, undecylene (—CH₂)₁₁—, dodecylene (—CH₂)₁₂—, or with at least one heteroatom, such as alkylene-CO—, based on the reaction of 10-undecenoic acid, 3-butenoic acid, acrylic acid, methacrylic acid and 5-hexenoic acid, alkylene-O(CO)-alkylene, alkylene-(CO)O-alkylene, alkylene-NH(CO)-alkylene, alkylene-(CO)NH-alkylene, alkylene-NH(CO)NH-alkylene, alkylene-NH(CO)O-alkylene, or from polyether radicals containing alkyl, aryl or alkyl and aryl groups and of the formula IVa or IVb where Q1′ and Q2′ are in each case independently

-T-O—(CH₂—CH₂—O—)ₓ—(CH₂—CH(R^{#})O—)_{y}—(SO)—R″ (IVa)

-T-O—(CH₂—CH₂—O—)ₓ—(CH₂—CH(R^{#})O—)_{y}—R″ (IVb),

where T=bivalent hydrocarbon radical with 2 to 4 carbon atoms, where x=0 to 200, in particular with 0 to 100, preferably with 0 to 50, y=0 to 200, in particular with 0 to 100, preferably with 0 to 50, where x and y are integers with the proviso that x or y is at least 1, where R^{#} is hydrogen or methyl, R″ is hydrogen or alkylene, in particular —(CH₂)₂—, —(CH(CH₃))CH₂—, methylene, polymethylene or —(CH₂)₃—, in particular —CH₂—CH₂—, preferably ethylene, preferably where T=—(CH₂)₂— or —(CH₂)₃—. With R″ hydrogen in a starting material or intermediate and linear or branched alkylene in an intermediate or end product.

Particularly preferred bivalent radicals -Q1″- and -Q2″- are isophorone derivatives, cyclohexylene containing radicals and polymethylene, such as hexamethylene.

Subsequent siloxane polymers are particularly preferred siloxane polymers and are selected from siloxane polymers of the formula Ia and Ib

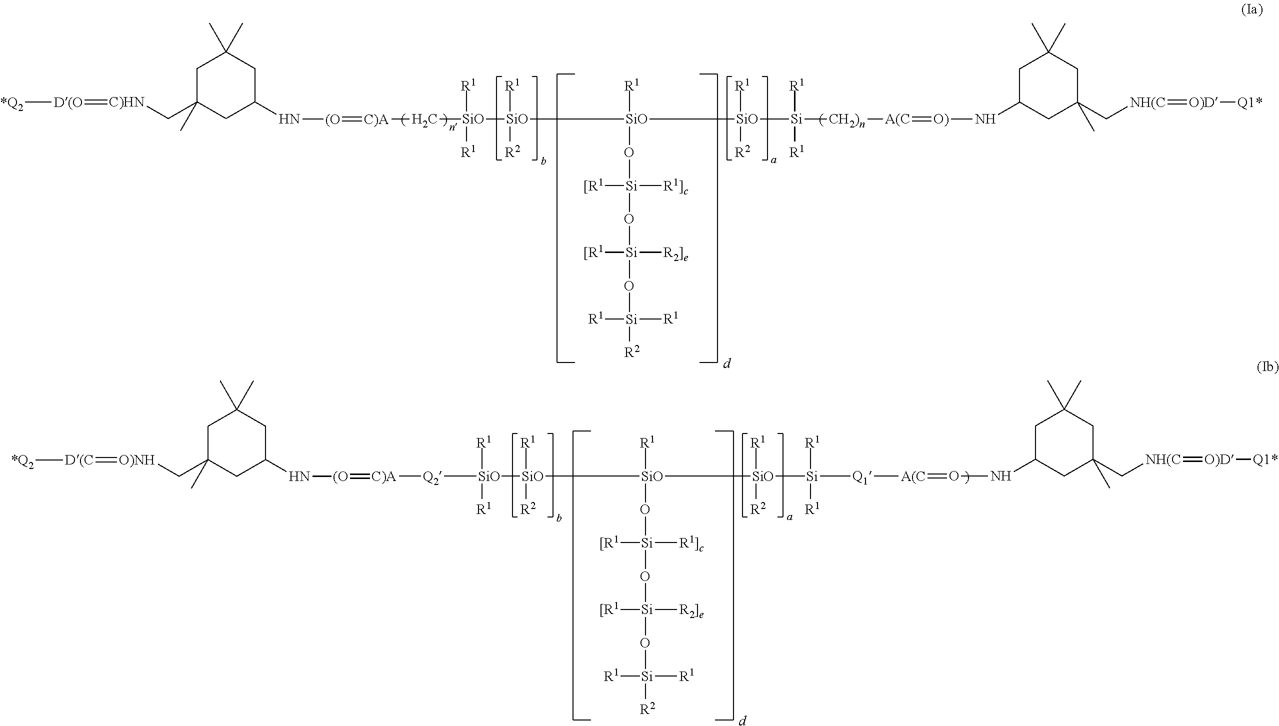

with n or n′ in each case independently selected from an integer from 2 to 40, in particular with 3 to 22, with a from 1 to 200, with b from 0 to 200, with c from 0 to 200, with d from 0 to 200, with e from 0 to 200 and with R¹ in formula Ia and Ib in each case independently identical or different, where R¹ comprises alkyl radicals with 1 to 4 carbon atoms or phenyl radicals, where R² is an alkyl radical with 1 to 22 carbon atoms, preferably 1 to 4 carbon atoms, in particular 1, 2, 3, 4, 6 carbon atoms, an alkyl radical with at least one heteroatom selected from N, O, S, preferably alkylamine, glycidyloxyalkyl radical or phenyl radical, in particular R² is an alkylamine or a radical obtained by reaction of an alkylamine with a coumarin isocyanate, in particular of the formula IX, preferably of the formulae IXa, IXb, IXc and/or IXd or mixtures of these, and where -D′-Q1* and -D′-Q2* is in each case independently derived from a hydroxy-coumarin, hydroxy-coumarin derivative or salts thereof, and in formula Ib where Q1′ and Q2′ in each case independently is a linear, cylic, branched alkylene with 2 to 40 carbon atoms or a formula IVa or IVb

-T-O—(CH₂—CH₂—O—)ₓ—(CH₂—CH(R^{#})O—)_{y}—(SO)—R″ (IVa)

-T-O—(CH₂—CH₂—O—)ₓ—(CH₂—CH(R^{#})O—)_{y}—R″ (IVb)

with T=bivalent hydrocarbon radical with 2 to 4 carbon atoms, where x=0 to 200, in particular with 0 to 150, 1 to 150, 5 to 150, 5 to 100, y=0 to 200, in particular with 0 to 150, 1 to 150, 5 to 150, 5 to 100, where x and y are integers with the proviso that x or y is at least 1, with R^{#} hydrogen or methyl, R″ hydrogen or alkylene, —(CH₂)₂—, —(CH(CH₃))CH₂—, methylene, polymethylene or —(CH₂)₃—, in particular —CH₂—CH₂—, preferably ethylene, in particular with T=—(CH₂)₂— or —(CH₂)₃—,

R² can in part have the meaning of the radicals R¹ and the other radicals R² can be independently of one another radicals of the formula Id, R²=-M-Z⁺ A⁻ (Ic), where radicals R² are in each case a radical of the formula -M-Z⁺ A⁻, Z⁺ is a radical of the formula Id

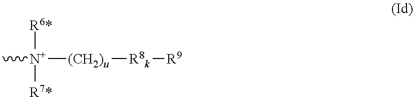

R^{6*}, R^{7*} * are in each case identical or different alkyl radicals with 1 to 22 carbon atoms or alkenyl radicals with 2 to 22 carbon atoms, in which the alkyl or alkenyl radicals can have hydroxyl groups,

where R⁸ is —O—(C═O)— or —NH(C═O)—,

R⁹ can be a monovalent hydrocarbon radical with 1 to 22 carbon atoms, or,

u=0 to 6 in formula Id,

k=0 or 1 in formula Id

M is a divalent hydrocarbon radical with at least 4 carbon atoms, which can have a hydroxyl group and which can be interrupted by one or more oxygen atoms,

A⁻ is an inorganic or organic anion which originates from a customary physiologically compatible acid HA,

One embodiment of the invention comprises siloxane polymers selected from siloxane polymers of the formula Ia* and Ib**

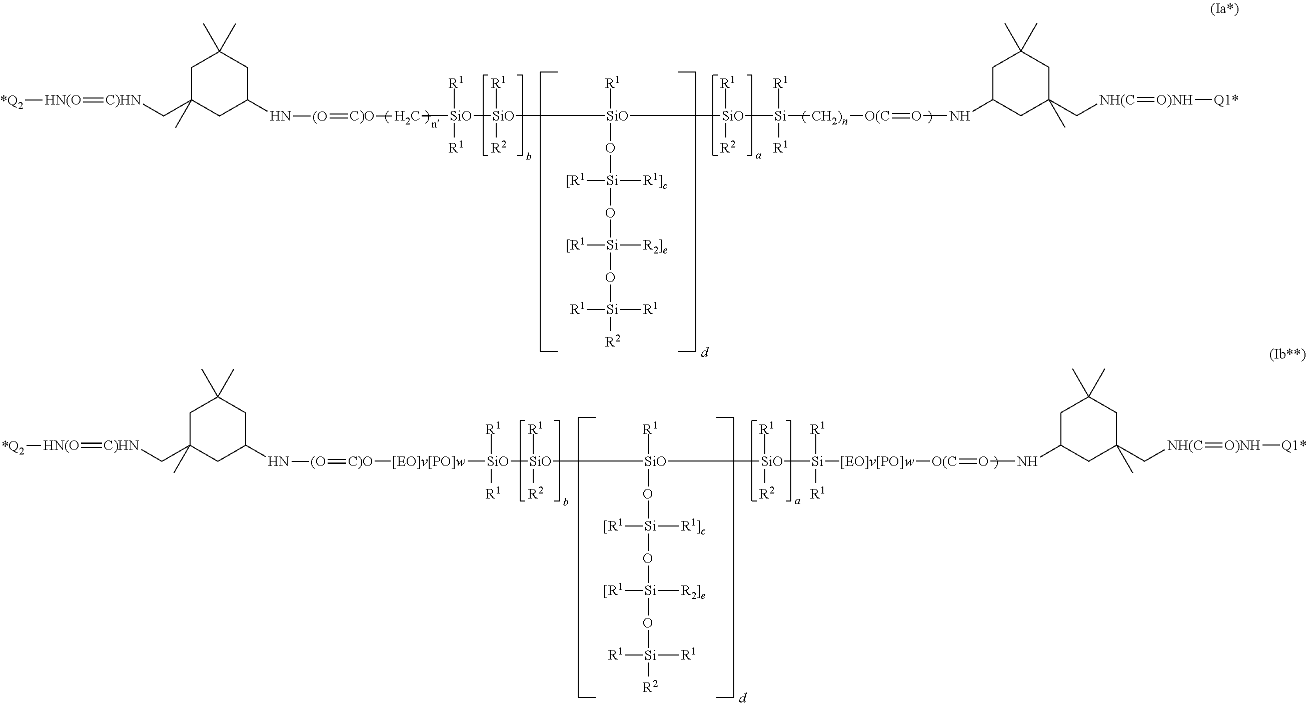

with n or n′ in each case independently selected from an integer from 3 to 22, with a from 1 to 200, in particular with a from 20 to 100, with b from 0 to 200, with c from 0 to 200, with d from 0 to 200, with e from 0 to 200, in each case as defined above, and with R¹ in formula Ia and Ib in each case independently identical or different, where R¹ comprises alkyl radicals with 1 to 4 carbon atoms or phenyl radicals, with R² alkyl radical with 1 to 22 carbon atoms, preferably 1 to 4 carbon atoms, in particular 1, 2, 3, 4 carbon atoms, an alkyl radical with at least one heteroatom selected from N, O, S, such as alkylamine, glycidyloxyalkyl radical, or phenyl radical and with Q1* and Q2* in each case independently coumarin or a coumarin derivative, in particular coumarin bonded to C-7, C-4 or C-6 of the coumarin or of a coumarin derivative or salts thereof, where the fragment —O-Q1* and —O-Q2* forms, by reaction of the hydroxy group of a coumarin with an isocyanate group, the carbamate group and in formula Ib** with [EO]v[PO]w, with v from 0 to 200 and w from 0 to 200, in particular in each case independently at least v or/and w is 5 to 100, or equally Q1′ and Q2′ are in each case independently equal to formulae IVa or IVb,

-T-O—(CH₂—CH₂—O—)ₓ—(CH₂—CH(R^{#})O—)_{y}—(SO)—R″ (IVa)

-T-O—(CH₂—CH₂—O—)ₓ—(CH₂—CH(R^{#})O—)_{y}—R″ (IVb),

where T=bivalent hydrocarbon radical with 2 to 4 carbon atoms, where x=0 to 200, y=0 to 200, preferably as defined above, preferably x and/or y is 5 to 100, where x and y are integers with the proviso that x or y is at least 1, with R^{#} hydrogen or methyl, R″ is hydrogen or alkylene, —(CH₂)₂—, —(CH(CH₃))CH₂—, methylene, polymethylene or —(CH)—, in particular —CH—CH—, and preferably ethylene, in particular with T=—(CH₂)₂— or —(CH₂)₃—. Where R″ in a starting material can correspond to hydrogen and in an intermediate or product to a linear or branched alkylene.

FIGS. 1 to 6 show by way of example the siloxane polymers of the formulae Ia, Ib, Ic obtainable by the process according to the invention, without limiting the invention to these examples. FIG. 1a shows a siloxane polymer of the general formula Ia, which is obtainable from the reaction with isophorone diisocyanate and a hydroxy- or amino-functional coumarin, such as umbelliferone, the linker is an alkylene with n or n′ in each case independently an integer between 2 and 40, A can be —O— or —NH—, —S—. FIG. 1b shows a specific compound with oxygen bridged coumarin, where linkers Q1′ and Q2′ can be an alkylene or a polyether. FIG. 2 shows a preferred embodiment of the general formula Ib*, which is likewise obtainable by a reaction with an isophorone diisocyanate and a hydroxy-functional UV chromophore, such as coumarin, and the second isocyanate group of the isophorone has been reacted with a hydroxy-functionalized siloxane, e.g. a hydroxyalkyl-functionalized siloxane. FIG. 3 defines more specifically in formula Ia* the formula Ia in that Q2′ and Q1′ are in each case a bivalent alkylene and A is —O—. FIG. 4 shows Ib** with a bivalent polyether -[EO]v[PO]w-, with v and w as defined above. The siloxane polymers of the formulae I can also be reacted with an aminoalkyl-functionalized siloxane to give a siloxane polymer with two urea groups and coumarin derivatives as terminal groups, see Example 3. FIG. 6 shows one possible isomer, if the process takes place by an alternative route of variant b) via the preparation of UV chromophore isocyanates, preferably coumarin isocyanates, by reacting a hydroxy-functional chromophore with IPDI to give a compound of the formula IX and then a reaction with a siloxane derivative of the formula VI (FIG. 6: Ic).

According to a particularly preferred alternative, it is likewise possible to use a siloxane of the general formula XI in the process according to the invention, in particular as explained below. In the process according to the invention R¹⁷ is then hydrogen or -Q1′-A-(C═O)-D-Q1″-NCO, -Q2′-A-(C═O)-D-Q2″-NCO or -Q1′-AH, -Q2′-AH.

A siloxane of the general formula XI as siloxane polymer according to the invention, in particular of the general formula I, is likewise obtainable by the process, where R¹⁷ are in each case independently -Q1 and -Q2 for a2 greater than or equal to 1, in particular with a2 greater than or equal to 2.

1) In general, preferably at least one siloxane of the general formula XI is obtainable by the process according to the invention with a) with R¹⁷ as defined for siloxane polymers in a).

2) Likewise, preferably at least one siloxane of the general formula XI can be used in the process with R¹⁷ corresponding to the definition in b) for the general formula XI

Mₐ₁M^{A}ₐ₂M^{B}ₐ₃D_{b1}D^{A}_{b2}D^{B}_{b3}T_{c1}T^{A}_{c2}T^{B}_{c3}Q_{d1} (XI)

where

M=[R¹⁶₃SiO_{1/2}]

M^{A}=[R¹⁷R¹⁶₂SiO_{1/2}]

M^{B}=[R¹⁸R¹⁶₂SiO_{1/2}]

D=[R¹⁶₂SiO_{2/2}]

D^{A}=[R¹⁷₁R¹⁶₁SiO_{2/2}]

D^{B}=[R¹⁸₁R¹⁶₁SiO_{2/2}]

T=[R¹⁶SiO_{3/2}]

T^{A}=[R¹⁷SiO_{3/2}]

T^{B}=[R¹⁸SiO_{3/2}]

Q=[SiO_{4/2}],

where

R¹⁶ are independently of one another identical or different linear or branched, saturated or unsaturated hydrocarbon radicals with 1 to 30 carbon atoms or else aromatic hydrocarbon radicals with 6 to 30 carbon atoms, preferably methyl or phenyl, in particular methyl,

a) for the siloxane polymer, in particular of the formula I, shown via the formula XI where R¹⁷ is in each case independently -Q1, -Q2 for a siloxane polymer of the general formula I, where the siloxane of the formula XI (without radicals R¹⁷, i.e. M^{A}=[—R¹⁶₂SiO_{1/2}],

D^{A}=[—R¹⁶₁SiO_{2/2}]) and/or T^{A}=[—SiO_{3/2}] corresponds to the fragment B of the formula I and the formula XI with R¹⁷ is equivalent to the formula I with Q2-B-Q1.

b) in the process for the preparation of the siloxane polymers: Alternatively, a siloxane of the formula XI with R¹⁷ can be used in the process for the preparation of at least one siloxane polymer, in particular of the formula I, with R¹⁷ comprising -Q1′-AH, -Q2′-AH, -Q1′-A-(C═O)-D-Q1″-NCO, OCN-Q2″-D-′(O═C)-A-Q2′-, hydrogen for Si—H group, —OH, —OR¹⁶, in particular —OMe, -AH, particular preferably in an alternative R¹⁷ is a saturated hydrocarbon radical with terminal —OH or —NH₂ group, preferably with 8 to 30, particularly preferably with 8 to 20 carbon atoms, in particular in M^{A} and optionally D^{A} or R¹⁷ is R¹⁸ in M^{B}, D^{B} and/or T^{B},

R¹⁸ independently of one another are identical or different linear or branched, saturated or olefinically unsaturated hydrocarbon radicals with 8 to 30 carbon atoms, for example decyl-, dodecyl, tetradecyl-, hexadecyl-, octadecyl-, in particular hexadecyl- and octadecyl-,

an aromatic hydrocarbon radical with 6 to 40 carbon atoms, an alkylaryl radical with 7 to 40 carbon atoms,

a linear or branched, optionally double-bond-containing aliphatic hydrocarbon radical with 2 to 30 carbon atoms interrupted by one or more heteroatoms (oxygen, NH, NR′ where R′ is an optionally double-bond-containing C1 to C30-alkyl radical, in particular —CH₃),

a linear or branched, optionally double-bond-containing aliphatic hydrocarbon radical with 2 to 30 carbon atoms interrupted by one or more functionalities selected from the group —OH

—O—C(O)—, —(O)C—O—, —NH—C(O)—, —(O)C—NH, —(CH₃)N—C(O)—, —(O)C—N(CH₃)—, —S(O₂)—O—, —O—S(O₂)—, —S(O₂)—NH—, —NH—S(O₂)—, —S(O₂)—N(CH₃)—, —N(CH₃)—S(O₂)—,

a terminally OH, OR′, NH₂, N(H)R′, N(R′)₂ (where R′ is an optionally double-bond-containing C1 to C30 alkyl radical) functionalized linear or branched optionally double-bond-containing aliphatic or cycloaliphatic hydrocarbon radical with 1 to 30 carbon atoms or

a blockwise or randomly structured polyether according to —(R⁵—O)ₙ—R⁶, where R⁵ is a linear or branched hydrocarbon radical containing 2 to 4 carbon atoms, n is 1 to 100, preferably 2 to 60, and R⁶ is hydrogen, a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms,

or a radical —C(O)—R⁷ where R⁷ is a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms, particularly preferably in an alternative R¹⁸ is a saturated hydrocarbon radical with terminal —NH₂ groups, preferably with 8 to 30 carbon atoms, particularly preferably with 8 to 20 carbon atoms,

where

a1=0-200, preferably 1-60, in particular 0,

a2=0-30, preferably 1-20, in particular 2-10, such as 2, 3, 4, 5, 6, 7, 8, 9, 10

a3=0-30, preferably 1-20, in particular 0, such as 1, 2, 2, 3, 4, 5, 6, 7, 8, 9, 10, 15, 20

b1=2 to 5000, preferably 10 to 1000, in particular 10-500, particularly preferably 2 to 100, preferably 10 to 100,

b2=0 to 100, preferably 1 to 30, in particular 1 to 10 or 0,

b3=0 to 100, preferably 0 to 30, in particular 1 to 10 or 0,

c1=0 to 30, preferably 1 to 30,

c2=0 to 30, preferably 0 to 5, in particular 0,

c3=0 to 30, preferably 0 to 5, in particular 0,

d1=0 to 30, preferably 0 to 5, preferably 0,

with the proviso that at least one of the indices selected from a1, a2 and a3 is not 0, in particular with (a2+b2+c2) greater than or equal to 1, preferably a2 is an integer between 2 and 10, preferably 2 to 5, such as 2, 3, 4 or 5, where it is further preferred that b1 is from 10 to 150, preferably 10 to 100. Optionally, additionally a1 and/or a3 can be an integer between 2 and 10, preferably 2 to 5, such as 2, 3, 4 or 5. With the proviso that (c1+c2+c3) is an integer greater than or equal to 1 if the sum of (a1+a2+a3) is an integer greater than 2. According to one alternative, a1 and/or a3 can also additionally be an integer greater than 1.

Siloxanes having at least one group selected from hydroxy and amino group that are used in the process and preferred according to the invention are characterized by the parameter characterization selected from the group:

a1=0, a2=2, a3=0, b1=10-100, b2=0, b3=0, c1=0, c2=0, c3=0 and d1=0, a1=0, a2=2, a3=0, b1=10-100, b2=1-30, b3=0, c1=0, c2=0, c3=0 and d1=0;

a1=0, a2=2, a3=0, b1=20-40, b2=1-30, b3=0, c1=0, c2=0, c3=0 und d1=0,

a1=0, a2=2, a3=0, b1=41-90, b2=1-30, b3=0, c1=0, c2=0, c3=0 and d1=0,

a1=0, a2=2, a3=0, b=5-350, b2=0, b3=0, c1=0, c2=0, c3=0 and d1=0,

a1=0, a2=2, a3=0, b1=15-200, b2=0, b3=0, c1=0, c2=0, c3=0 and d1=0,

a1=0, a2=2, a3=0, b1=10-150, b2=0, b3=1 to 5, c1=0, c2=0, c3=0 and d1=0;

a1=0, a2=0, a3=2, b=5-350, b2=0, b3=0, c1=0, c2=0, c3=0 and d1=0,

a1=0, a2=0, a3=2, b1=15-200, b2=0, b3=0, c1=0, c2=0, c3=0 and d1=0,

a1=2, a2=0, a3=2 to 5, b1=10-150, b2=1-30, b3=0, c1≧0, c2≧0, c3≧0 and d1=0; where (c1+c2+c3) is greater than or equal to 1 to 3

a1=0, a2=2, a3=0, b1=10-150, b2=0, b3=1-2, c1=0, c2=0, c3=0 and d1=0,

a1=0, a2=2, a3=0, b1=51-90, b2=0, b3=1-2, c1=0, c2=0, c3=0 and d1=0,

a1=0, a2=0, a3=2, b1=10-50, b2=0, b3=1-2, c1=0, c2=0, c3=0 and d1=0,

a1=0, a2=1, a3=1, b1=10-150, b2=0, b3=1 to 5, c1=0, c2=0, c3=0 and d1=0;

The index numerals a, b, c, d, e, f, a1, a2, a3, b1, b2, b3, c1, c2, c3, d1, d2, d3, v, w, n, n′ etc. in formulae I, II, III, IV, XI and all of the associated substructures, which are named for example with Arabic letters, and the value ranges of the stated indices are understood to be averages of the possible statistical distribution of the actually present structures and/or mixtures thereof. This is also true for structural formulae exactly reproduced as such per se, such as for example for formula I, II, III and III, or IIa, IIb, IIIa, IIIb.

Statistical distributions can be blockwise in structure with any desired number of blocks and any desired sequence or be subject to a randomised distribution, they can also have an alternating structure or else form a gradient via the chain, in particular they can also form all mixed forms in which optionally groups of different distributions can follow one another. Specific embodiments can lead to the statistical distributions experiencing limitations due to the embodiment. For all regions which are not affected by the limitation, the statistical distribution is not changed.

Preferably, R¹⁷ in M^{A} and/or D^{A} optionally T^{A} is selected from the two following formulae IX1 and IX2 or comprise a radical of the formulae XIIa or XIIb.

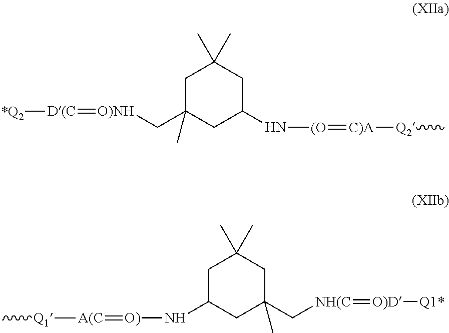

In addition to the UV chromophore isocyanates, such as coumarin isocyanates of the general formulae (IXa to IXd) it is also possible to react further substituted isocyanate derivatives, preferably from the reaction with diamines comprising one tertiary and one primary amino-groups on hydrocarbons optionally comprising O or N in the reaction with a reactive hydroxy- or amino-functional siloxane, e.g. of the formula VI. The reaction can also take place in a mixture comprising coumarin isocyanates. In formulae IX1, IX2 where Z=-Q1*, Q2*, as derived from formula XIII or an amine, a diamine, such as DMPAPA, etc.

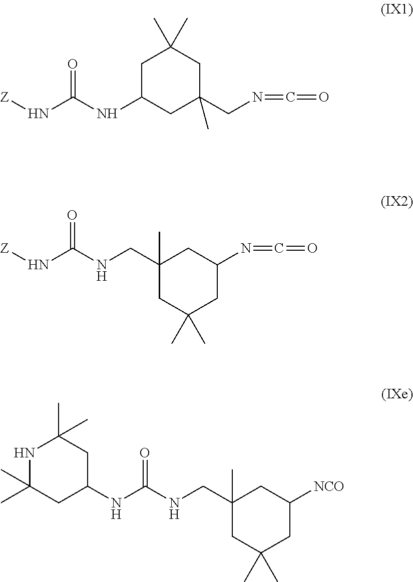

According to one alternative, in addition to the reaction with hydroxy-coumarin or a hydroxy-coumarin derivative, an additional reaction with sterically hindered amines with a primary amino or hydroxy group can take place, particular preference being given to the diamines with a sterically hindered nitrogen as basic group, particularly preferably HALS amine of the formula XIII (4-amino-2,2,6,6-tetramethylpiperidine) or N,N-dimethylaminopropylamine (DMAPA) or 3-(dimethylamino)propylamines (CAS: 109-55-7), N-(3-aminopropyl)imidazoles (CAS: 5036-48-6), dimethylethanolamine (CAS: 108-01-0), dimethylaminoethoxyethanol (CAS: 1704-62-7); trimethylaminoethylethanolamine (CAS: 2212-32-0) or salts thereof. Also conceivable is a reaction of lateral, functional groups or Si—OH groups with one of the aforementioned amines, preferably of the formula XIII.

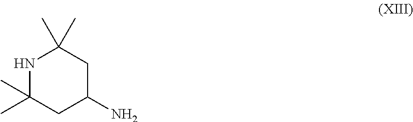

The invention likewise provides a process for the preparation of a siloxane polymer, in particular of the formula I, preferably of the formula XI, and siloxane polymers and compositions comprising these siloxane polymers obtainable by the process with a central polysiloxane polymer block B, in particular a process for the preparation of at least one siloxane polymer of the general formula (I), as described above, and of compositions comprising these siloxane polymer or mixtures of the siloxane with a central polysiloxane polymer block B, by reacting a) a polysiloxane diisocyanate of the formula VII,

OCN-Q2″-D-(O═C)-A-Q2′-B-Q1′-A-(C═O)-D-Q1″-NCO (VII)

with at least one hydroxy- or amino-functional UV/Vis chromophore or salt thereof, where the reaction preferably takes place in the molar ratio of at least 1:1 with regard to the isocyanate groups of the polysiloxane to amino or hydroxy groups of the chromophores,

and a siloxane polymer of the general formula (I)

Q2-B-Q1 (I)

is obtained, where -Q1 corresponds to the general formula IIa and -Q2 corresponds to the formula IIb,

-Q1=-Q1′-A-(C═O)-D-Q1″-A′-(C═O)-D′-Q1* (IIa)

-Q2=-Q2′-A-(C═O)-D-Q2″-A′-(C═O)-D′-Q2* (IIb)
where A is —NH—, —O— or —S— and D is —NH— in each case independently in formulae IIa and IIb,
where A′ is —NH— and D′ is —NH—, —O— or —S— in each case independently in formulae IIa and IIb, where each radical Q1 and Q2 of the formula IIa or IIb has in each case independently at least two bivalent groups selected from carbamate and urea group, in particular each radical -Q1 and -Q2 has two carbamate groups or a carbamate and a urea group or else two urea groups, or reacting

b) a polysiloxane of the formula VI

HA-Q2′-B-Q1′-AH (VI)

with a UV/Vis chromophore isocyanate, in particular a UV chromophore isocyanate selected from coumarin isocyanates, selected from the formulae IXa, IXb, IXc and IXd

Q2*-O(CO)NH-″2Q-NCO (IXa)

Q1*-O(CO)NH-″1Q-NCO (IXb)

Q2*-O(CO)NH-Q2″-NCO (IXc)

Q1*-O(CO)NH-Q1″-NCO (IXd)
where A is —NH—, —O— or —S— and D is —NH— in each case independently in formulae VII,
I, VI, IXa, IXb, IXc and IXd, in particular comprising IXa and IXb,
with Q1′ and Q2′ in each case independently comprising a bivalent hydrocarbon radical with 6 to 200 carbon atoms optionally comprising at least one heteroatom comprising O, N or S, a bivalent radical comprising aryl, arylalkyl groups or a bivalent radical
comprising aryl, arylalkyl groups optionally comprising at least one heteroatom O, N or S or polyether radicals containing alkyl, aryl or alkyl and aryl groups, in each case independently in formulae VII, I and/or VI,
with Q1″ and Q2″ in each case independently comprising a bivalent linear, branched and/or cyclic alkyl radical with 4 to 200 carbon atoms, in particular a cyclic C6 alkyl radical with alkyl side chains, or a bivalent radical comprising an aryl and/or arylalkyl radical with 6 to 200 carbon atoms, in each case
independently in formulae VII, IVa and/or IVb,
with —O-Q1* or —O-Q2* for d′-Q1*, D′-Q2* in each case independently as -Q1* and -Q2*
a UV/Vis chromophore as radical, in particular a UVA2 chromophore,
in particular with at least one absorption maximum from 320 to 340 nm,
and/or UVB chromophore, in particular with at least one absorptions maximum of 280-320 nm, which preferably comprises a coumarin, coumarin
derivative or a salt thereof.

FIG. 9 shows an obtainable diisocyanate according to formula VII with D in each case —NH—. Preferably, (i) the hydroxy- or amino-functional UV/Vis chromophore is a UV chromophore comprising hydroxy-coumarin, amino-coumarin or a derivative of coumarin, ii) the UV/Vis chromophore isocyanate is a UV chromophore, selected from coumarin isocyanates from the formulae IXa, IXb, IXc and IXd, with —O-Q1* and —O-Q2* in each case independently as radical -Q1* and/or -Q2* coumarin or coumarin derivative, and/or (iii) in formula IIa, IIb, IXa, IXb, IXc and/or IXd and I —O-Q1* and —O-Q2* comprise in each case independently radicals from the reaction of hydroxycoumarin or hydroxycoumarin derivative, in particular 7-hydroxycoumarin, 4-hydroxycoumarin, 6-hydroxycoumarin or hydroxy-isocoumarin with a diisocyanate. The reaction in step a) can proceed in the presence of a catalyst, such as the catalysts known in the prior art for polyurethane preparation and isocyanate trimerization. By way of example, tertiary amines such as triethylamine, tetraethylenediamine, or strong bases such as DBU, and tin and bismuth compounds, such as for example dibutyltin laurate or tin(II) octoate are mentioned.

According to one process variant, the formulae IVa, IVb and I comprise as —ONH-Q1* and

—O-Q2* in each case independently radicals which are derived from a hydroxy-functional 7-coumarin, 4-coumarin, 6-coumarin.

The process according to the invention can in particular comprise the following steps and comprise individual steps:
(I) H—B—H (x)+Q1′-AH (VIIIa), Q2′-AH (VIIIb)→HA-Q2′-B-Q1′-AH (VI)
(II) HA-Q2′-B-Q1′-AH+diisocyanate→OCN-Q2″-D-(O═C)-A-Q2′-B-Q1′-A-(C═O)-D-Q1″-NCO (VII)
   where -AH is —NH₂ or —OH
(III) OCN-Q2″-D-(O═C)-A-Q2′-B-Q1′-A-(C═O)-D-Q1″-NCO (VII)+coumarin derivative→Q2-B-Q1 (I)
   or alternatively
(Ia) diisocyanate (e.g. IPDI)+hydroxy-coumarin derivative→Q2*-O(CO)NH-″2Q-NCO (IXa)+Q1*-O(CO)NH-″1Q-NCO (IXb) and optionally Q2*-O(CO)NH-Q2″-NCO (IXc) and/or Q1*-O(CO)NH-Q1″-NCO (IXd)
(Ib) H—B—H (x)+Q1′-AH (VIIIa), Q2′-AH (VIIIb)→HA-Q2′-B-Q1′-AH (VI)
(II) HA-Q2′-B-Q1′-AH (VI)+Q2*-O(CO)NH-″2Q2-NCO (IXa)/Q1*-O(CO)NH-″1Q-NCO (IXb)→Q2-B-Q1 (I)

To prepare the siloxane polymers, firstly

(i) a polysiloxane-group-containing linear and/or branched polymer block B, in particular of the formulae IIIa and/or IIIb or of the formula XI with R¹⁷═H and a2 is greater than or equal to 2, b1 is greater than or equal to 1 or a1 is greater than or equal to 1, a2 is greater than or equal to 1 and b1 is greater than or equal to 1, with at least two terminal Si—H groups or at least one terminal Si—H group and at least one lateral Si—H group, e.g. H—B—H(X), where —H corresponds to two Si—H groups, can be reacted

(ii) with an olefinic compound comprising alkylene and optionally at least one heteroatom such as N, O, S, in particular alkenylenol, alkylenamine, alkylenecarboxylic acid, alkylene ester, alkylenamide or an olefinic polyether is reacted, where the olefinic compound has in each case independently an allyl or vinyl group and corresponds to the formulae VIIIa and/or VIIIb

Q1′-AH (VIIIa)

Q2′-AH (VIIIb)

with Q1′ and Q2′ in each case independently comprising an alkenylene with 6 to 200 carbon atoms optionally comprising at least one heteroatom O or N, aryl or arylalkyl groups optionally comprising at least one heteroatom O or N, olefinic polyether with -AH in formulae VIIIa and VIIIb are independently selected from —OH and —NH₂. The reaction preferably takes place in (iii) in the presence of a catalyst, such as a Karstedt catalyst, to give a polysiloxane of the formula VI,

HA-Q2′-B-Q1′-AH (VI)

where in each case independently in formulae VIIIa, VIIIb and VI with AH independently selected from —OH and —NH₂, and with -Q2′- and -Q1′- in each case independently comprising a bivalent hydrocarbon with radical 6 to 200 carbon atoms optionally comprising at least one heteroatom O or N, a bivalent radical comprising aryl, arylalkyl groups optionally comprising at least one heteroatom O or N or olefinic polyether.

For the reaction of olefinic compounds with the Si—H group, hydrosilylation catalysts are used. The use of a Karstedt catalyst is customary. Generally, preference is given to platinum catalysts in which platinum(0) is present.

Mercaptoalkyl-substituted siloxanes, in particular of the formula I, VI or XI can be prepared by the person skilled in the art by processes known to him from the prior art via a condensation and/or equilibration.

In a subsequent process step, the polysiloxane of the formula VI

HA-Q2′-B-Q1′-AH (VI)

where A is selected from —O, —NH, —S— or AH selected from —OH, —NH₂, and —SH with -Q2′- and -Q1′- as defined above, are reacted with a diisocyanate to give a polysiloxane diisocyanate of the formula VII, preferably the diisocyanate is IPDI,

OCN-Q2″-D-(O═C)-A-Q2′-B-Q1′-A-(C═O)-D-Q1″-NCO (VII)

with -Q2″- and/or -Q1″- independently selected from a bivalent, linear, branched and/or cyclic alkyl radical with 4 to 200 carbon atoms, in particular an isophorone radical, or a bivalent radical comprising an aryl and/or arylalkyl radical with 6 to 200 carbon atoms, where the molar ratio of HA groups in the polysiloxane to isocyanate groups is at least 1:1, in particular the ratio is 1:100 to 1:1, preferably 1:10 to 1:1.

Particular preference is given to reacting a diisocyanate with a hydroxy-functional UV/Vis chromophore, in particular hydroxy-functional UV chromophore to give a UV chromophore isocyanate, e.g. according to the formulae IXa and IXb.

In the next process step, the prepared polysiloxane diisocyanate of the formula VII or any desired polysiloxane diisocyanate of the formula VII prepared by a different process

OCN-Q2″-D-(O═C)-A-Q2′-B-Q1′-A-(C═O)-D-Q1″-NCO (VII)

where B is a linear and/or branched polysiloxane polymer block B,

with -Q2′- and -Q1′- in each case independently comprising a bivalent hydrocarbon radical with 6 to 200 carbon atoms optionally comprising at least one heteroatom O, N or S, a bivalent radical comprising aryl, arylalkyl groups optionally comprising at least one heteroatom O, N or S, or polyether radicals containing alkyl, aryl or alkyl and aryl groups, where A is in each case independently —NH—, —O— or —S— and D is —NH— in each case independently in formula VII, and with -Q2″- and/or -Q1″- independently selected from a bivalent, linear, branched and/or cyclic alkyl radical with 4 to 200 carbon atoms, or a bivalent radical comprising an aryl and/or arylalkyl radical with 6 to 200 carbon atoms,

can be reacted with a hydroxy- or amino-functional UV/Vis chromophore, in particular a hydroxy-functional UV chromophore, preferably with at least one absorption maximum in the range from 280 to 380 nm and optionally with at least one absorption maximum from 300 to 380 nm, preferably a hydroxy-functional coumarin, coumarin derivative or a salt thereof.

Preference is given to hydroxy-functional UVA2 chromophores, in particular with at least one absorption maximum in the range from 320 to 340 nm and optionally a further absorption maximum in the range from 280 to 320, and/or a hydroxy-functional UVB chromophore, in particular with at least one absorption maximum in the range from 280-320 nm, preferably an absorption maximum in the range from 280 to 300 and optionally a further from 301 to 320 nm. Particular preference is given to hydroxy-functional coumarin, coumarin derivative or a salt thereof.

In the process according to the invention, difunctional isocyanates selected from the group comprising for example; toluene 2,4-diisocyanate (TDI), diphenylmethane diisocyanate or methylenediphenyl diisocyanate (MDI), hexamethylene diisocyanate (HMDI), 2,2,4-trimethylhexane 1,6-diisocyanate (TMDI), polymeric diphenylmethane diisocyanate (PMDI), isophorone diisocyanate (IPDI), 4,4′-diisocyanatodicyclohexylmethane (H12MDI) can be used, preference being given to the aliphatic products, and isophorone diisocyanate (IPDI) being particularly preferred.

Some of these isocyanates have stereocentres. In particular, reference is made to the isomers of isophorone. Expressly, all conceivable isomers are included within the scope of this invention. Thus, for example, isophorone diisocyanate can be differentiated into a cis and a trans isomer. Particular preference is given to isophorone diisocyanate of a cis/trans mixture of 5:1 to 1:5, preferably 3:1 to 1:3, further preferably 1:1. A particularly preferred, commercial product consists of a cis/trans mixture of 3:1. The use of commercial isophorone diisocyanate is preferred. Isophorone diisocyanate is obtainable under different names, which are included as synonyms within the scope of this invention: 3-isocyanatomethyl-3,5,5-trimethylcyclohexyl isocyanate, 5-isocyanato-1-(isocyanatomethyl)-1,3,3-trimethylcyclohexane, CA RN: 4098-71-9. Various trade names are customary, often they contain the name of the starting molecule isophorone, although other trade names are also customary: e.g. Desmodur®I (BAYER), Isocur IPDI 22-200 (ISO-ELEKTRA), VESTANAT® IPDI (EVONIK INDUSTRIES), which are likewise included within the scope of the invention. Customary specifications for isophorone diisocyanate are: Total chlorine content <400 mg/kg, hydrolysable chlorine <200 mg/kg, purity >99.5% by weight, refractive index n²⁵_{D} 1.483 (DIN 51 423, Part 2), NCO content 37.5-37.8% by weight (EN ISO 11 909/ASTM D 2572), the commercial product is described as colourless to slightly yellow. The specified isocyanates can optionally at least partially comprise prepolymers.

Suitable isocyanate-group-containing compounds are all known isocyanates. Within the context of the teaching according to the invention, preference is given to e.g. aromatic, aliphatic and cycloaliphatic polyisocyanates with a number-average molar mass of less than 800 g/mol. Thus, of suitability are for example diisocyanates selected from the series 2,4-/2,6-toluene diisocyanate (TDI), methyldiphenyl diisocyanate (MDI), triisocyanatononane (TIN), naphthyl diisocyanate (NDI), 4,4′-diisocyanatodicyclohexylmethane, 3-isocyanatomethyl-3,3,5-trimethylcyclohexyl isocyanate (isophorone diisocyanate=IPDI), tetramethylene diisocyanate, hexamethylene diisocyanate (HDI), 2-methylpentamethylene diisocyanate, 2,2,4-trimethylhexamethylene diisocyanate (THDI), dodecamethylene diisocyanate, 1,4-diisocyanatocyclohexane, 4,4′-diisocyanato-3,3′-dimethyldicyclohexylmethane, 4,4′-diisocyanatodicyclohexylpropane(2,2), 3-isocyanatomethyl-1-methyl-1-isocyanatocyclohexane (MC1), 1,3-diisooctylcyanato-4-methylcyclohexane, 1,3-diisocyanato-2-methylcyclohexane and [alpha],[alpha],[alpha]′,[alpha]′-tetramethyl-m- or -p-xylylene diisocyanate (TMXDI), and mixtures consisting of these compounds.

Preferred starting materials for the preparation of the urethane groups and preferably of the compounds containing urea groups are isophorone diisocyanate (IPDI) and/or 4,4′-diisocyanatodicyclohexylmethane.

Preference is given to using the following hydroxy-coumarin or hydroxy-coumarin in the process, and in particular the hydroxy group is reacted with an isocyanate group. In the reaction with diisocyanates, a coumarin isocyanate is obtained.

The invention also provides compositions obtainable by the process according to the invention, in particular comprising siloxane polymers with at least two carbamate groups, a urea and a carbamate group or two urea groups per organofunctional radical and comprising a UV chromophore.

According to a further embodiment, the invention provides a composition comprising a) siloxane polymers of the general formula XI and mixtures of these or b) siloxane polymers with a central polysiloxane polymer block B selected from (i) at least one siloxane polymer of the general formula I, and mixtures comprising this polymer,

(ii) at least one siloxane polymer of the general formula Ia, and mixtures comprising this polymer or

(iii) at least one siloxane polymer of the general formula Ib, and mixtures comprising this polymer, and of the formula Ia* and/or Ib* and also mixtures thereof or mixtures comprising these.

The invention further provides an intermediate for the preparation of siloxane polymers, in particular of the formula I, selected from coumarin isocyanates selected from the formulae IXa, IXb, IXc and IXd, in particular IXa*, IXb*, and optionally IXc* and IXd*, or salts thereof or mixtures of the coumarin derivatives.

Q2*-O(CO)NH-″2Q-NCO (IXa)

Q1*-O(CO)NH-″1Q-NCO (IXb)

Q2*-O(CO)NH-Q2″-NCO (IXc)

Q1*-O(CO)NH-Q1″-NCO (IXd)

With -″2Q- and -″1Q- with the meaning that the secondary isocyanate groups has reacted with the hydroxy group of the coumarin and the primary —CH₂—NCO group can later react with a polysiloxane of the general formula (VI) HA-Q2′-B-Q1′-AH, in particular where B is formula IIIa or IIIb and alternatively in the meaning of the formula XI where R¹⁷ in each case independently is -Q1′-AH or HA-Q2′- can be reacted.

With Q2*, Q2″, Q1* and Q1″, as defined above, where Q2* and Q1* are in each case independently coumarin or a coumarin derivative, in particular coumarin bonded to C-7, C-4 or C-6 of the coumarin or of a coumarin derivative, preferably coumarin isocyanates of the formulae IXa* and IXb* or salts thereof.

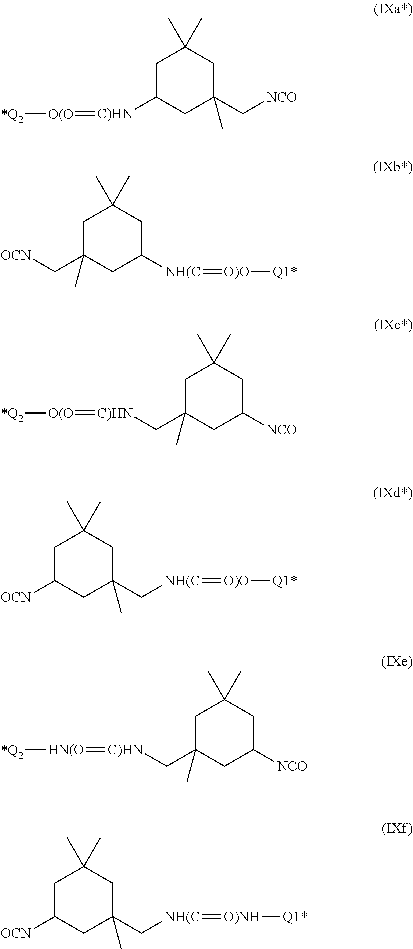

with a diamine, in particular of the formula XIII HALS amine or N,N-dimethylaminopropylamine (DMAPA) or 3-(dimethylamino)propylamine (CAS: 109-55-7), N-(3-aminopropyl)imidazole (CAS: 5036-48-6), dimethylethanolamine (CAS: 108-01-0), dimethylaminoethoxyethanol (CAS: 1704-62-7); trimethylaminoethylethanolamine (CAS: 2212-32-0) or salts thereof, N,N-dimethylaminopropylamine (DMAPA).

The siloxane polymers according to the invention of the invention should have advantageous properties as regards their UV activity. Of particular interest are siloxanes which are equipped with chromophore groups in order to prevent damage of natural surfaces, such as the skin or of hair, by having an absorption maximum in the UV region close to the transition of UVB to UVA at 315. An additive with these properties would be able to protect hair against UV damage without weighing it down. Moreover, such a siloxane, in particular with basic amino groups, would be able to position itself on the hair surface and, by virtue of the UV chromophore, protect the hair against damage by solar irradiation. According to the invention, the polysiloxane with 7-hydroxycoumarin, 4-hydroxycoumarin or 6-hydroxycoumarin is bonded via a diisocyanate to the siloxane and serves as protection against solar irradiation in the range around 315 nm. A lateral umbelliferone bonded via a urea and carbamate group to siloxane polymers is able to be used as UV protection in the region of about 327 nm in haircare products.

In order now to determine the absorption maximum at a certain wavelength of the compound 32 provided with the chromophoric substituents, it was dissolved in chloroform and measured in a UV spectrometer. The values obtained were normalized and are shown in FIG. 7a. As can be clearly seen, the PDMS-umbelliferone (32) absorbs strongest at 286 and 315 nm. In the literature 315 nm is defined inter alia as being the limit between the so-called UV-B and UV-A region. Compound 32 absorbs light accordingly precisely in this limit region and therefore corresponds to the above-described requirements for use as UV absorber in haircare products.

In addition to a UV protection, it was also a request to develop siloxanes which are able to protect the hair against free radicals. Suitable compounds are basic amines with sterically hindered amino groups such as HALS or DMAPA radicals, which can be bonded to the siloxanes according to the invention.

The invention therefore provides a formulation comprising at least one siloxane polymer or a mixture comprising at least one siloxane polymer or siloxane polymer prepared via the intermediate and at least one auxiliary. Preferably, the formulation is a cosmetic rinse for hair, care skin or hair product, lacquer, hair spray, hair colorants, colour, mouthwash, pharmaceutical formulation, impression material (technical, pharmaceutical, cosmetic, dental), cleaner, wood care product, paint care.

The invention further provides the use of the siloxane polymers, of the obtainable compositions comprising siloxane polymers by the process according to the invention as additive in cosmetic formulations, as additive in pharmaceutical formulations, in paints, pastes, as foam stabilizer or foam additive for polyurethane foams, in particular polyurethane rigid foams and polyurethane flexible foams, as hand improvers or impregnating agents during the production of fibres, textiles, in cosmetic formulations for the treatment, post-treatment and protection of keratin fibres, in particular in hair-conditioning formulations and skin and skin appendages, as additive in detergents, fabric softener formulations, in cosmetic formulations including creams, rinses, hair washing compositions, washing compositions, setting agents, care rinses, care pastes, sprays, hairsprays, for improving the combability of keratin or textile fibres of natural or synthetic origin.

The present invention further provides the use of the siloxanes according to the invention and/or of the siloxanes obtainable by the process according to the invention for producing formulations, in particular of care and cleaning formulations for use in the domestic and industrial sector. Preferred care and cleaning formulations for use in the domestic and industrial sector are in this connection textile care compositions, such as for example fabric softeners, and care compositions for hard surfaces.

The general synthesis of an isocyanate-terminated PDMS takes place by reacting hydroxy-terminated PDMS of different chain lengths, such as (n=30 or 80) (The specific compounds are illustrated in more detail in the preparation examples; 14 and 15) with isophorone diisocyanate (16), which is particularly recommended on account of its differently reactive isocyanate groups for a functionalization of the PDMS. In this way, it is possible to exclude the diisocyanate component from reacting twice with the hydroxyl groups of the PDMS and there being no free isocyanate group available for a further reaction with a substituent. Furthermore, gelation of the reaction mixture caused by the formation of high molecular masses can be prevented. For this, the isophorone diisocyanate (16) is introduced firstly in a secured apparatus and, with the addition of catalytic amounts of triethylamine, reacted without dilution with α,ω-bis(hydroxyhexyl)polydimethylsiloxane (PDMS-30 or PDMS-80) (14 and 15) added dropwise.

Wherever reference is made within the scope of this invention to natural substances, e.g. amino acid, in principle all isomers are intended, preference being given to the naturally occurring isomers in each case, in the case specified here thus the alpha-amino acids. As regards the definition of natural substances, reference is made to the scope of the “Dictionary of Natural Products”, Chapman and Hall/CRC Press, Taylor and Francis Group, e.g. in the online version from 2011: http://dnp.chemnetbase.com/.

Wherever molecules or molecule fragments have one or more stereocentres or can be differentiated into isomers on account of symmetries or can be differentiated into isomers on account of other effects e.g. restricted rotation, all possible isomers are included by the present invention. Isomers are known to the person skilled in the art, reference being made in particular to the definitions by Prof. Kazmaier of the University of Saarland, e.g. http://www.uni-saarland.de/fak8/kazmaier/PDF_files/vorlesungen/Stereochemie%2Strassb%20Vorlage.pdf. In particular, all options which arise from the stereochemical definitions of tacticity are included, e.g. isotactic, syndiotactic, heterotactic, hemiisotactic, atactic. Within the context of the invention, preference is given to polyethers and polyether fragments with at least partial atactic substituent sequence.

The following examples illustrate the siloxane polymers according to the invention and also the process according to the invention in more detail without limiting the invention to these examples.

### BRIEF DESCRIPTION OF THE DRAWINGS

FIGS. 1 to 6 show by way of example the siloxane polymers of the formulae Ia, Ib, Ic obtainable by the process according to the invention, without limiting the invention to these examples.

FIG. 1a shows a siloxane polymer of the general formula Ia, which is obtainable from the reaction with isophorone diisocyanate and a hydroxy- or amino-functional coumarin, such as umbelliferone, the linker is an alkylene with n or n′ in each case independently an integer between 2 and 40, A can be —O— or —NH—, —S—.

FIG. 1b shows a specific compound with oxygen bridged coumarin, where linkers Q1′ and Q2′ can be an alkylene or a polyether.

FIG. 2 shows a preferred embodiment of the general formula Ib*, which is likewise obtainable by a reaction with an isophorone diisocyanate and a hydroxy-functional UV chromophore, such as coumarin, and the second isocyanate group of the isophorone has been reacted with a hydroxy-functionalized siloxane, e.g. a hydroxyalkyl-functionalized siloxane.

FIG. 3 defines more specifically in formula Ia* the formula Ia in that Q2′ and Q1′ are in each case a bivalent alkylene and A is —O—.

FIG. 4 shows Ib** with a bivalent polyether -[EO]v[PO]w-.

FIG. 5 shows a specific compound with nitrogen bridged coumarin, where linkers Q1′ and Q2′ are in each case a bivalent alkylene and A is —O—; D′ is defined with formula (IIa & b).

FIG. 6 shows one possible isomer, if the process takes place by an alternative route of variant b) via the preparation of UV chromophore isocyanates, preferably coumarin isocyanates, by reacting a hydroxy-functional chromophore with IPDI to give a compound of the formula IX and then a reaction with a siloxane derivative of the formula VI (FIG. 6: Ic).

FIG. 7a shows UV-spectrum of umbelliferone-terminated polydimethylsiloxane according to example 2 (chloroform).

FIG. 7b shows UV spectrum of AP-PDMS-IPDI-Umbelliferon according to example 3 (chloroform).

FIG. 8a shows Structural formula of umbelliferone-terminated PDMS.

FIG. 8b shows UV spectrum (ethanol).

FIG. 9 shows an obtainable diisocyanate according to formula VII with D in each case —NH—.

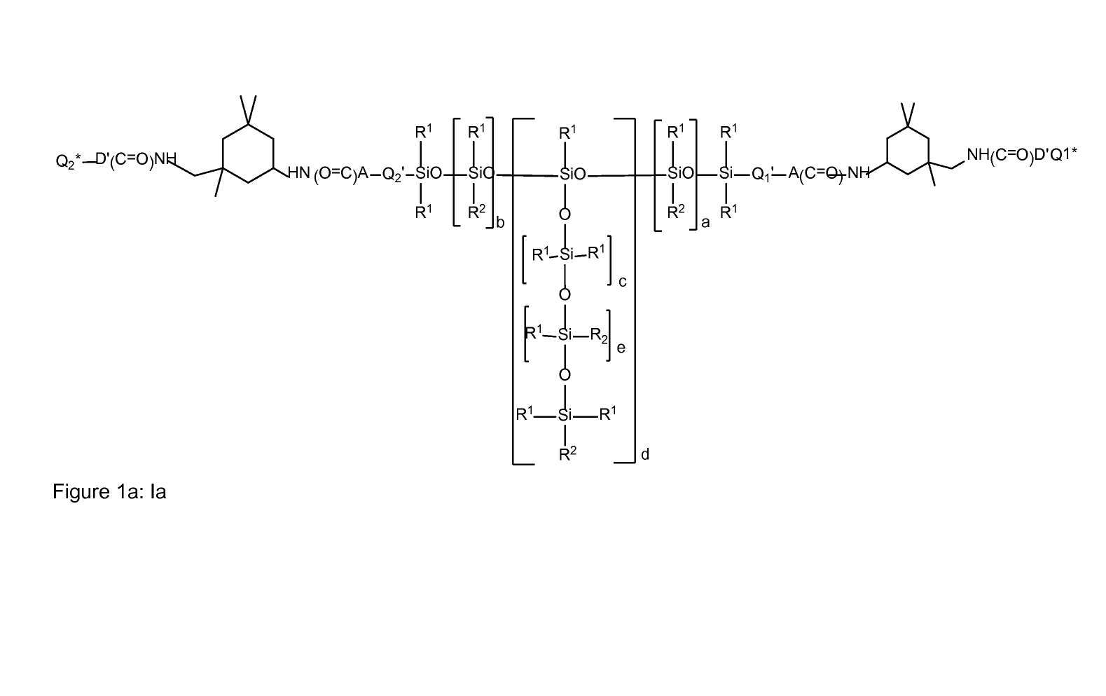

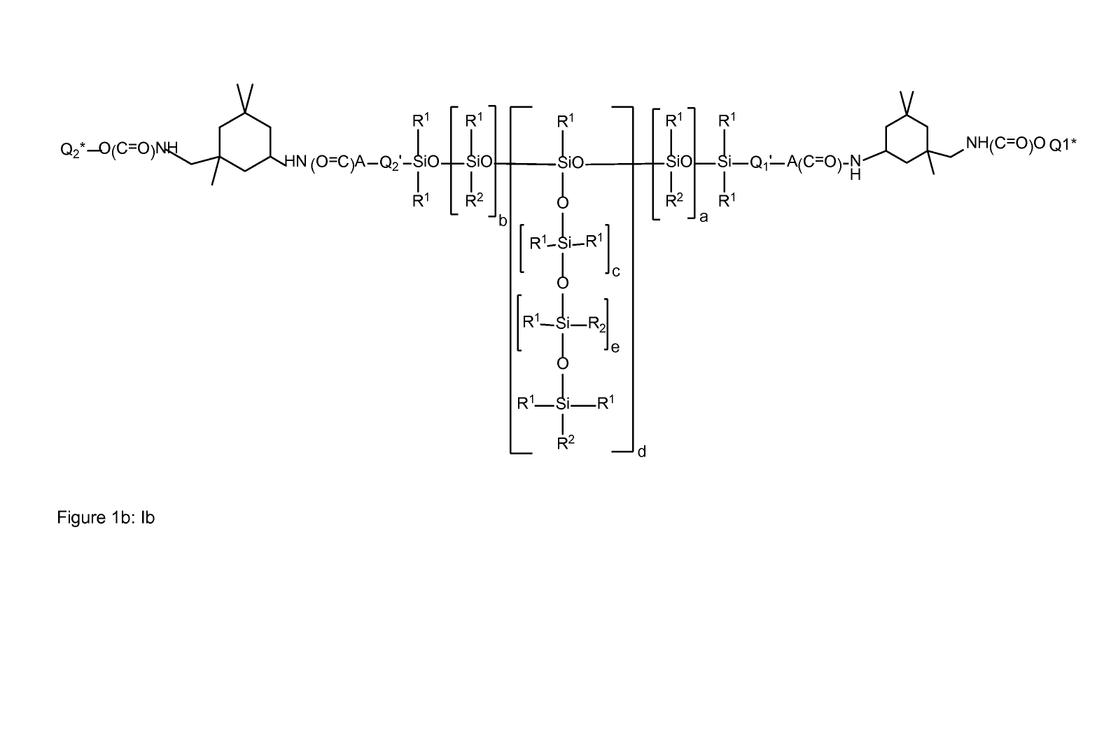

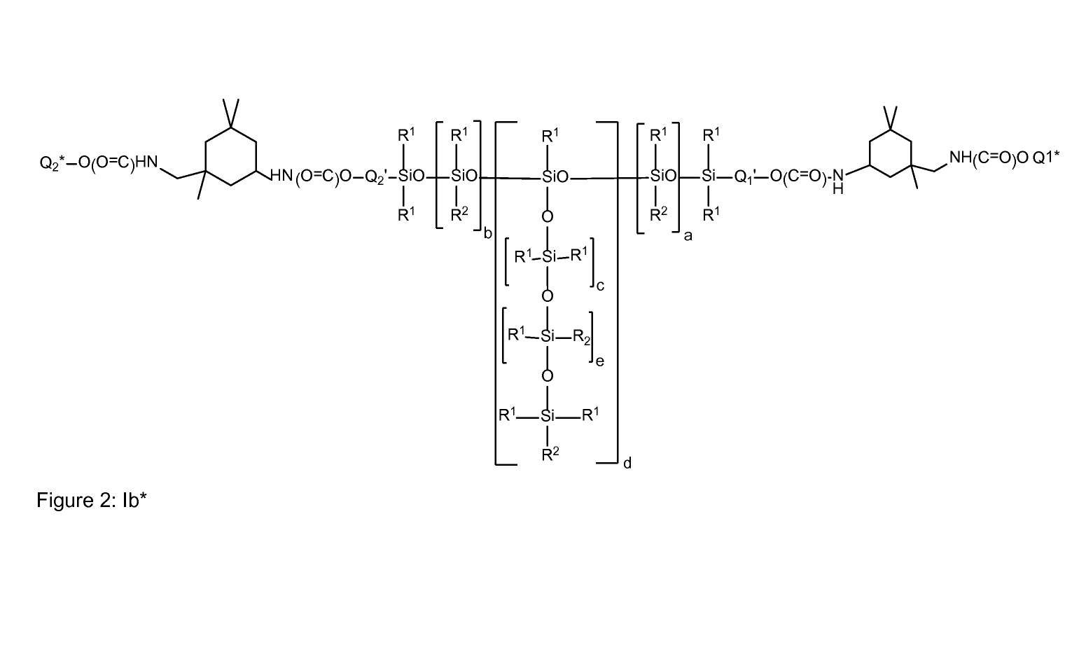

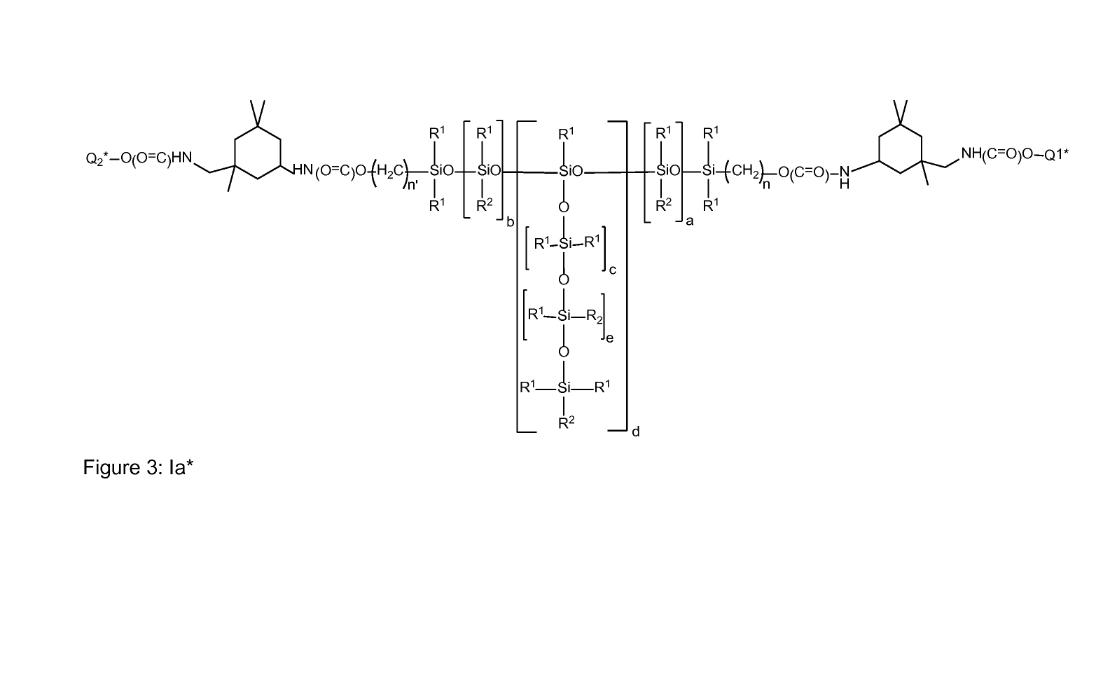

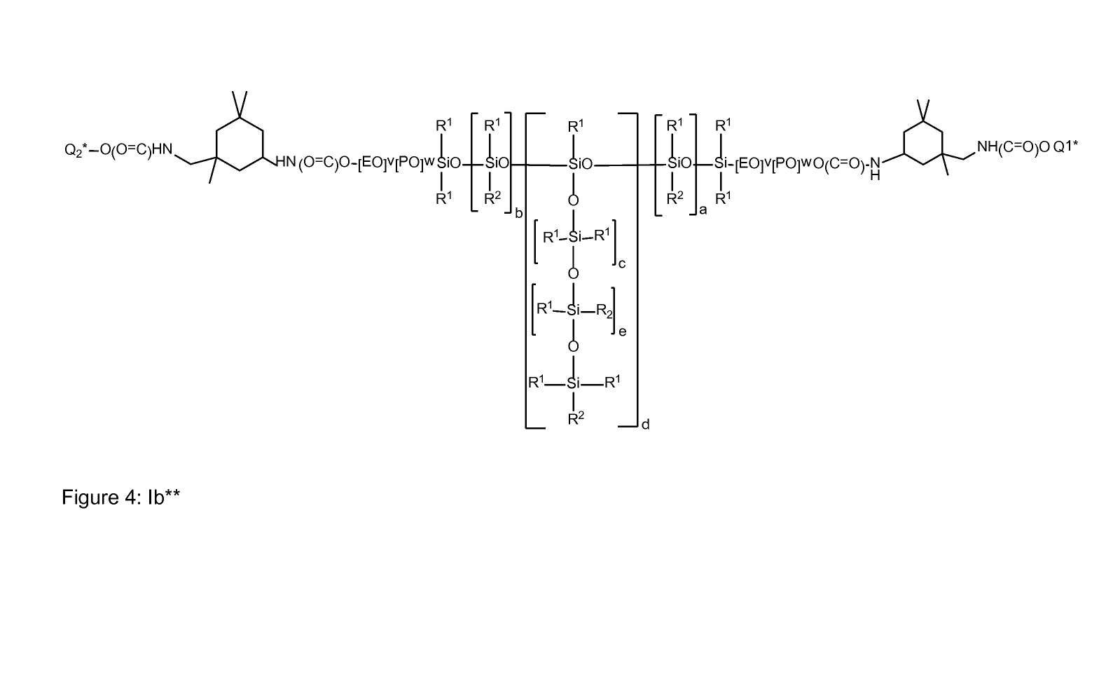

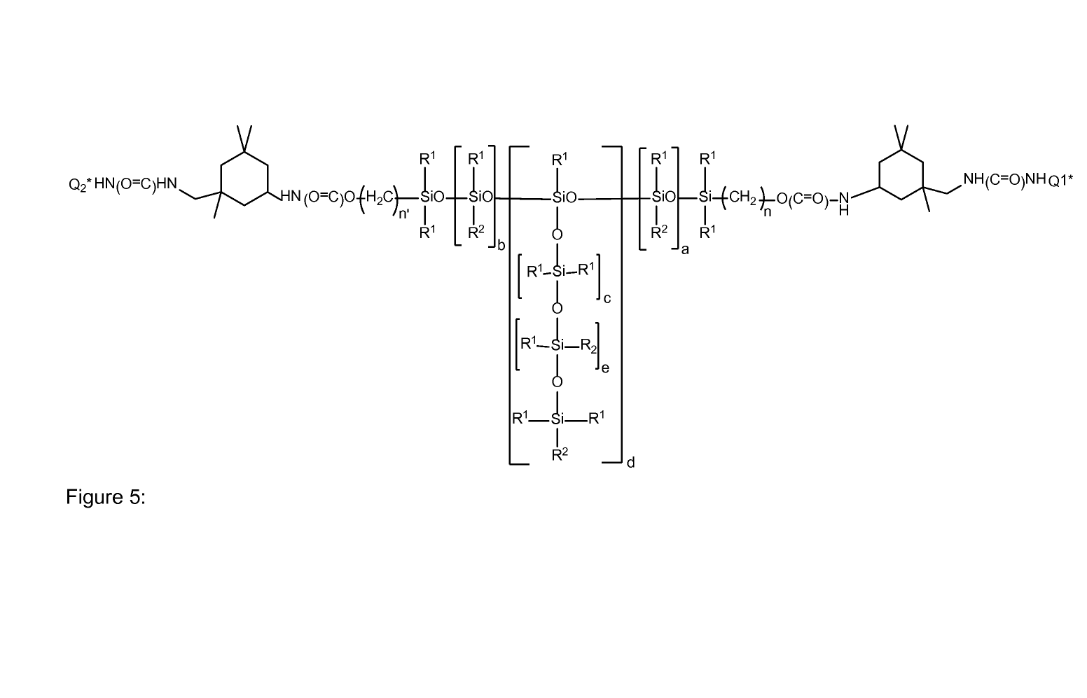

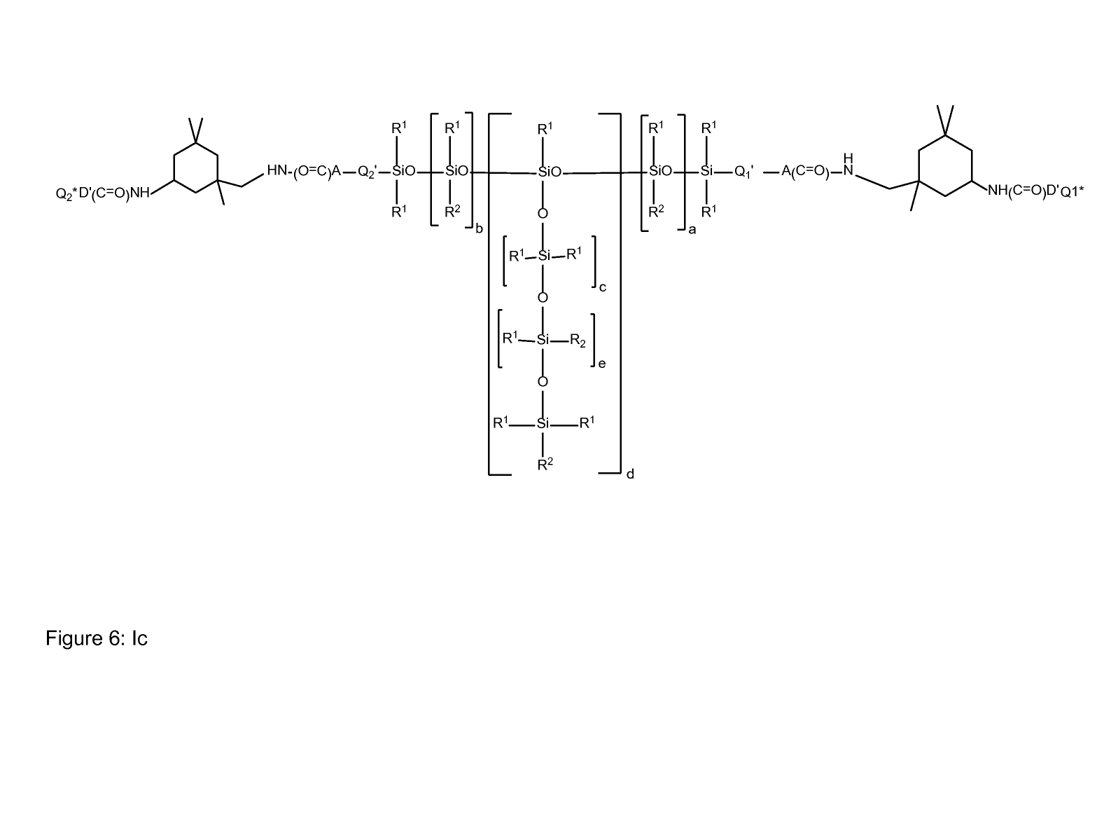

### OPERATIVE EXAMPLES

The subject matter of the present invention is elucidated in more detail below, without any intention that the subject matter of the invention should be confined to these exemplary embodiments.

Analysis:

UV-VIS spectroscopy: A double-beam spectrometer from Analytik Jena AG, model Specord© 210 Plus was used for the UV-VIS spectroscopic measurements. To avoid particle contaminations of the samples, they were filtered by means of an injection filter of pore size 0.45 μm and transferred to a quartz glass cuvette of path length 1 cm. The measurements were carried out in a range from 250 to max. 800 nm and at a temperature of 25° C.

MALDI-TOF-MS: Ultraflex time of flight-Mass spectrometer, Bruker. 337 nm nitrogen laser, linear mode or reflector mode. Weighed samples were dissolved in a suitable solvent. The matrix used was dithranol (DIT) or 2,5-dihydroxybenzoic acid (DHB).

FT-IR spectra: FT-IR-5SXB, Nicolet used. Calibration: by means of HeNe laser.

ATR measurements: specac golden-gate diamond ATR unit.

NMR spectroscopy: 300 MHz-NMR spectrometer, Bruker, model Avance III -300, magnetic field strength 7.05 Tesla. Absorption frequency: ¹H-NMR at 300 MHz, 13C{1H}-NMR at 75 MHz. 200 or 500 MHz-NMR spectra: FT-NMR spectrometer, Bruker DRX200 or DRX500.

### Example 1

### Synthesis of isocyanate-terminated polydimethylsiloxanes

The synthesis of an isocyanate-terminated PDMS was carried out by reacting hydroxy-terminated PDMS of different chain lengths (n=30 or 80) (14 or 15) with isophorone diisiocyanate (16). The advantage of the IPDI is that the diisocyanate component does not react twice with the hydroxyl groups of the PDMS. Furthermore, it was possible to avoid a gelation of the reaction mixture caused by the formation of high molecular masses. Isophorone diisocyanate (16) is placed in a secured apparatus and catalytic amounts of triethylamine are added with the dropwise-added α,ω-bis(hydroxyhexyl)polydimethylsiloxane (PDMS-30 or PDMS-80) (14 or 15) reacted without dilution. The reaction of the α,ω-bis(hydroxyhexyl)polydimethylsiloxanes 14 (n=30) and 15 (n=80) with isophorone diisocyanate (16) to give the α,ω-bis[hexyl(3-(isocyanatomethyl)-3,5,5-trimethylcyclohexyl)carbamyl]polydimethylsiloxanes 17 and 18 (PDMS-30-IPDI, n=30 or PDMS-80-IPDI, n=80) can preferably take place in the presence of dichloromethane at 60° C. A customary reaction time is 2 hours.

Analysis of isocyanate-terminated PDMS: MS, 1H-NMR, IR spectroscopy, molar masses (MALDI-TOF) confirm compounds 17 and 18; IR: characteristic C—H stretching and deformation vibrations of the polydimethylsiloxane at 2961, 1412 and 1257 cm−1, 2256 cm−1 free isocyanate groups, 1709 cm−1 C═O stretching vibration urethane unit. The reaction of PDMS-30-IPDI, n=30 (17) with umbelliferone (31) to give an umbelliferone-terminated polydimethylsiloxane (PDMS-Umb) (32) can preferably take place in a mixture of dichloromethane/DMF at room temperature. A customary reaction time is about 24 hours.

The product 32 was then analyzed by means of 1H-NMR and IR spectroscopy and MALDI-TOF. A MALDI-TOF spectrum of PDMS-Umb (32) was determined. The mass numbers higher intensity correspond here to the polydimethylsiloxane derivative, disubstituted with umbelliferone, of different chain lengths, which is present ionized with sodium. The distance between these mass numbers can in turn be assigned precisely to a siloxane unit. Consequently, the result of the mass spectroscopic analysis points to the successful synthesis of the structure 32. This is once again confirmed by reference to IR data. A stretching vibration of a free isocyanate group of the starting material 17 is no longer detected, which suggests complete conversion of the starting materials 17 and 31 to the desired product 32. Furthermore, the 1H-NMR spectroscopic analysis demonstrates the formation of the umbelliferone-terminated PDMS (32).

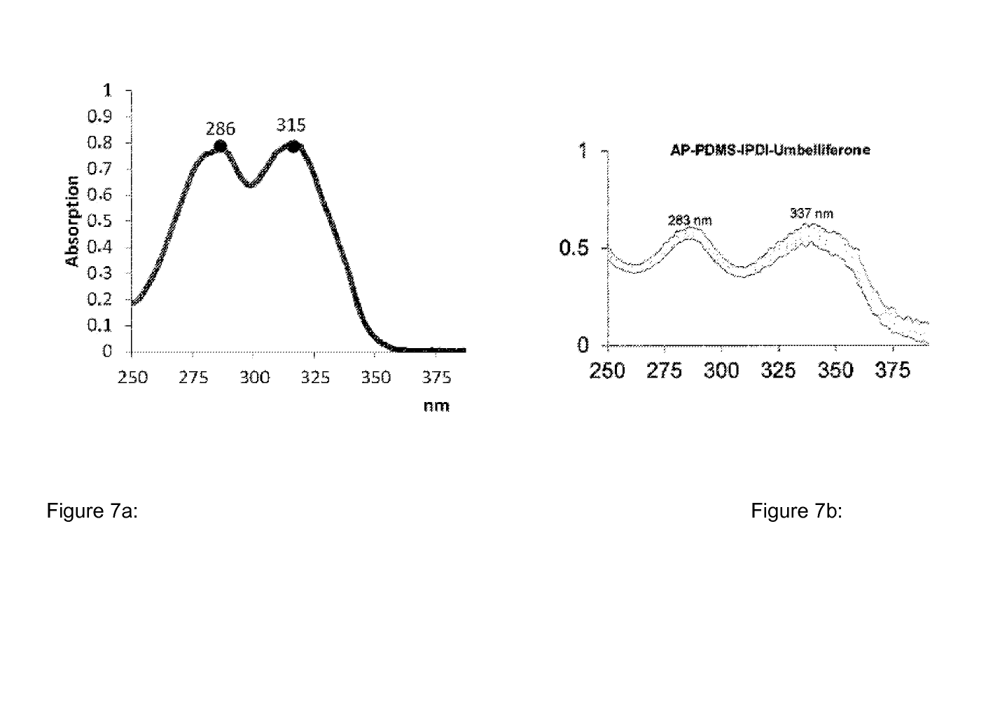

In order to now determine the absorption maximum at a specific wavelength of the compound provided with the chromophoric substituents 32, it was dissolved in chloroform and measured in a UV spectrometer. The values obtained were normalized and are shown in FIG. 7a. As can be seen clearly, the PDMS-Umb (32) absorbs greatest at 286 and 315 nm. In the literature, 315 nm is sometimes defined as the limit between the so-called UV-B and UV-A region. Compound 32 accordingly absorbs light precisely in this border range and therefore corresponds to the above-described requirements for use as UV absorber in haircare products.

### Example 2

### Synthesis of an umbelliferone-terminated polydimethylsiloxane (PDMS-umbelliferone) (32)

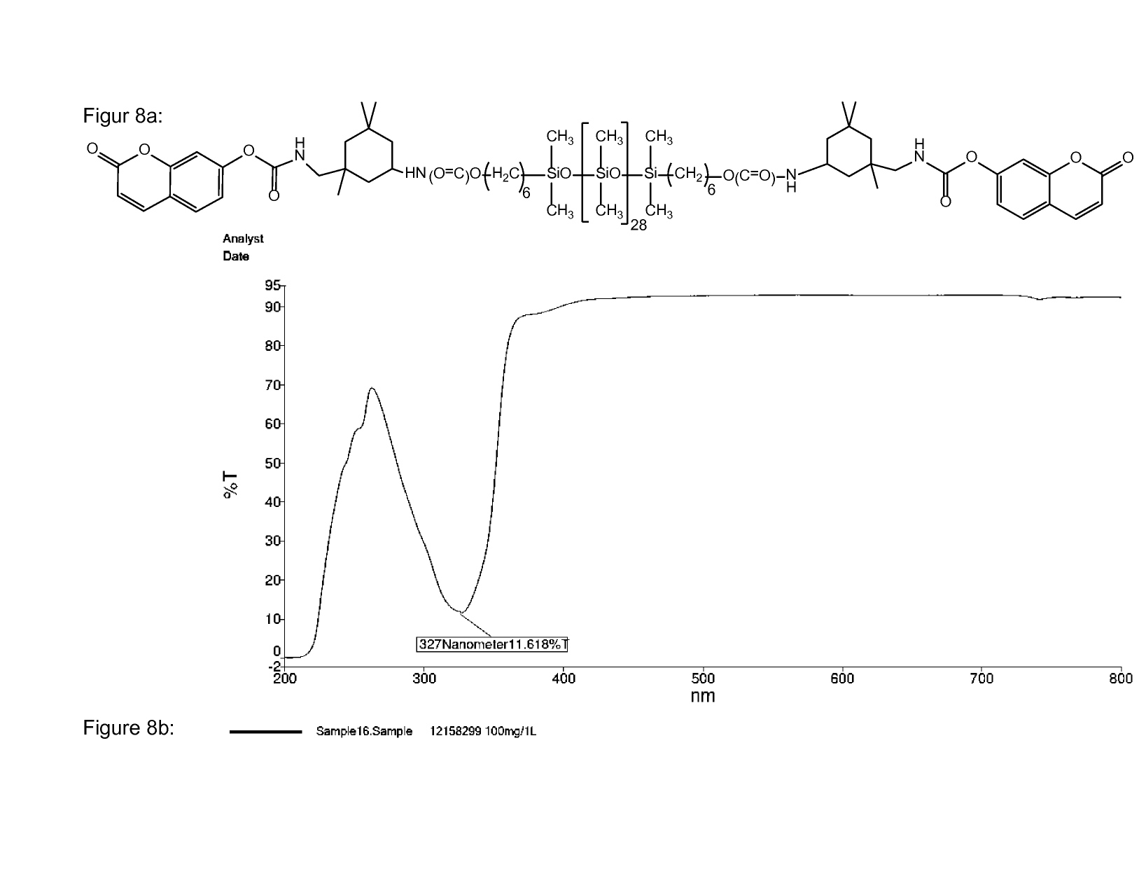

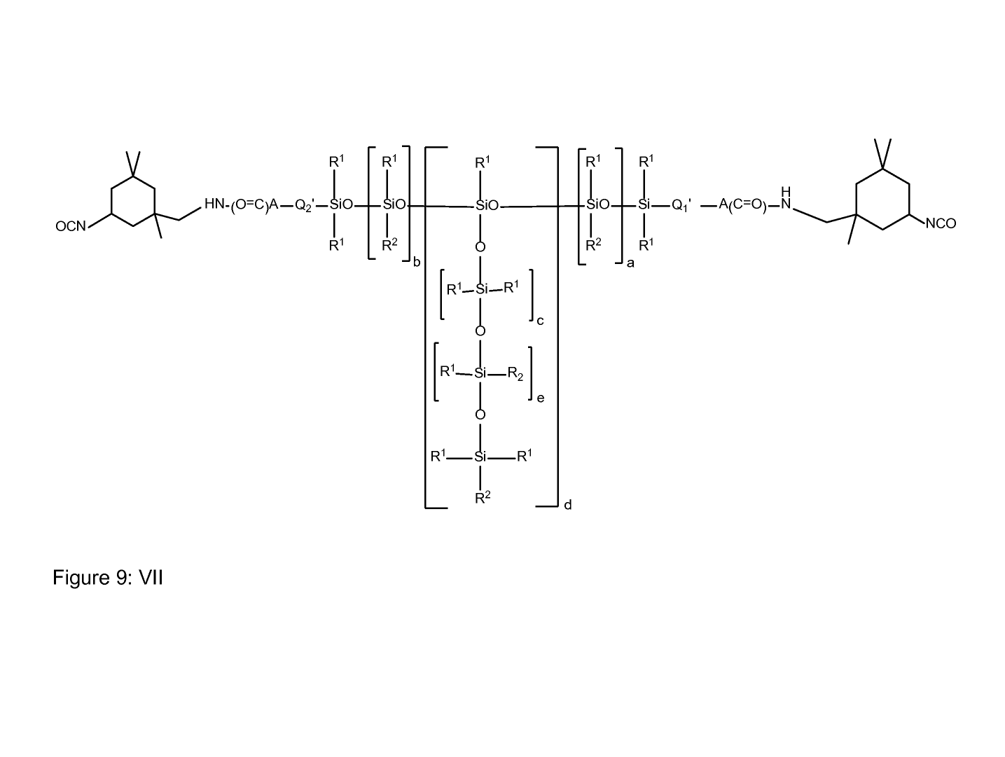

Structural formula of umbelliferone-terminated PDMS: FIG. 8a.

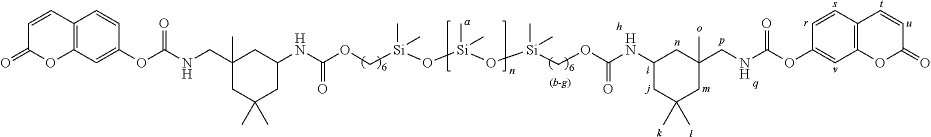

7-Hydroxycoumarin (0.39 g, 2.4 mmol) is dissolved in a secured round-bottomed flask including reflux condenser and dropping funnel in a mixture of 20 mL of dichloromethane and 20 mL of dimethylformamide. Triethylamine (0.5 mL) is added. α,ω-Bis[hexyl(3-(isocyanatomethyl)-3,5,5-trimethylcyclohexyl)carbamyl]-poly(dimethylsiloxane) (n=30) (3.4 g, 1.2 mmol) is likewise dissolved in a mixture of 20 mL of dichloromethane and 20 mL of dimethylformamide and added dropwise per dropping funnel with stirring over the course of 1 h. After stirring for 24 h at 40° C., washing is performed with 2×20 mL of dist. water and 1×20 mL of sat. aqueous sodium hydrogencarbonate solution. The organic phases are combined, dried over sodium sulphate and the solvent is removed on a rotary evaporator. The crude product is dissolved in some ethanol, precipitated again in dist. water and centrifuged. Finally, the product is dried in high vacuum.

1H-NMR (600 MHz, CDCl3, 24° C.): δ [ppm]=7.59 (2H, m, H-t), 7.39 (2H, m, H-s), 7.07 (4H, m, H-r/H-v), 6.32-6.15 (2H, m, H-u), 3.98 (4H, m, H-g), 3.76 (2H, m, H-i), 2.86 (4H, m, H-p), 1.97-1.44 (8H, m, H-f/H-n), 1.26 (12H, m, H-c/H-d/H-e), 1.15-0.66 (26H, m, H-j/H-k/H-l/H-m/H-o), 0.46 (4H, m, H-b), 0.00 (144H, m, H-a)

FT-IR (diamond): \tilde{∪} [cm−1]=2960 (∪ R—CH3, Si—CH3, m-w), 1666 (∪ C═C, RCO—O—CH═CH—R, s), 1530 (∪ C═O, urethane, m-w), 1437 (δ C—H, Si—CH3, w), 1257 (δ C—H, siloxane, s-m), 1089 (∪ Si—O—Si, siloxane, s-m)

MALDI-TOF-MS m/z: 1505 [M+Na]+ (for n=7), disubstituted

UV spectrum (chloroform): FIG. 7a

UV spectrum (ethanol): FIG. 8b

100 mg of the compound were dissolved in 1 l of ethanol and measured in a UV spectrometer. The transmissions minimum (=absorption maximum) of the compound from Example 2 is at 327 nm.

### Example 3

### Synthesis of an umbelliferone-functionalized aminopropyl-polydimethylsiloxane copolymer (AP-PDMS-IPDlumbelliferone) (36)

U-IPDI (24.0 mg, 0.06 mmol) is introduced in a previously secured apparatus at RT in 10 mL of dichloromethane. A dropping funnel is then used to add the aminopropyl-polydimethylsiloxane copolymer (10.0 g, 2.0 mmol), which was likewise dissolved beforehand in 40 mL of dichloromethane, over the course of 1 h dropwise with an argon countercurrent. After stirring for 24 h at RT, the solvent is removed on a rotary evaporator and the product is finally dried in a high vacuum.

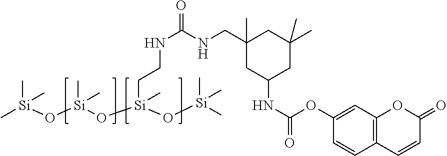

FT-IR (diamond): \tilde{∪} [cm−1]=2962 (∪ R—CH3, Si—CH3, m-w), 2901 (∪ C—H, —CH2-, m-w), 1590 (∪ C═C, aromatic, v) (∪ C═O, N—CO—N, v), 1444 (δ C—H, S1-CH3, w), 1412 (δ C—H, Si—CH3, m-w), 1257 (δ C—H, siloxane, s-m), 1009 (∪ Si—O—Si, siloxane, s-m)

UV spectrum: FIG. 7b

### Example 4

### Synthesis of short-chain α,ω-bis[hexyl(3-(isocyanatomethyl)-3,5,5-trimethylcyclohexyl)carbamyl]polydimethylsiloxane (PDMS-30-IPDI) (17), n=30

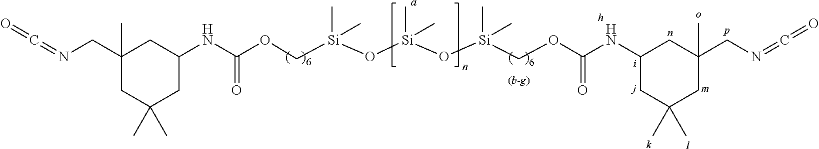

Isophorone diisocyanate (4.72 mL, 22.5 mmol) is introduced in a secured round-bottomed flask with reflux condenser and dropping funnel. Heating to 60° C. is performed under a protective gas atmosphere and with stirring. 0.1% by weight of triethylamine (32 mg) are added. Over the course of 2 h, α,ω-bis(hydroxyhexyl)poly(dimethylsiloxane) (n=30) (26.4 g, 11.0 mmol) is slowly added dropwise. Stirring is continued until there is no more clouding. The product is obtained in quantitative yield.

1H-NMR (300 MHz, CDCl3, 24° C.): δ [ppm]=3.98 (4H, m, H-g), 3.57 (2H, m, H-i), 2.98 (4H, m, H-p), 1.88-1.38 (8H, m, H-f/H-n), 1.26 (12H, m, H-c/H-d/H-e), 1.20-0.72 (26H, m, H-j/H-k/H—I/H-m/H-o), 0.46 (4H, m, H-b), 0.00 (168H, m, H-a); FT-IR (diamond): \tilde{∪} [cm−1]=2961 (∪ R—CH3, Si—CH3, m-w), 2256 (∪ —NCO, isocyanate, s), 1709 (∪ C═O, urethane, s), 1412 (δ C—H, Si—CH3, w), 1257 (δ C—H, siloxane, s-m), 1011 (∪ Si—O—Si, siloxane, s-m); MALDI-TOF-MS m/z: 1542 [M+Na]+ (for n=10), disubstituted

### Example 5

### Synthesis of long-chain α,ω-bis[hexyl(3-(isocyanatomethyl)-3,5,5-trimethylcyclohexyl)carbamyl]polydimethylsiloxane (PDMS-80-IPDI) (18), structure analogous to Example 4 where n=80

Isophorone diisocyanate (4.72 mL, 22.5 mmol) is introduced in a secured round-bottomed flask together with reflux condenser and dropping funnel. Heating to 60C is performed under a protective gas atmosphere and with stirring. 0.1% by weight of triethylamine (32 mg) are added. α,ω-Bis(hydroxyhexyl)poly(dimethylsiloxane) (n=80) (67.1 g, 11.0 mmol) is slowly added dropwise over the course of 2 h. Stirring is continued until there is no more clouding. The product is obtained in quantitative yield.

1H-NMR (300 MHz, CDCl3, 24° C.): δ [ppm]=4.01 (4H, m, H-g), 3.77 (2H, m, H-i), 3.01 (4H, m, H-p), 1.89-1.42 (8H, m, H-f/H-n), 1.33 (12H, m, H-c/H-d/H-e), 1.24-0.76 (26H, m, H-j/H-k/H—I/H-m/H-o), 0.51 (4H, m, H-b), 0.00 (396H, m, H-a); FT-IR (diamond): \tilde{∪} [cm−1]=2960 (∪ R—CH3, Si—CH3, m-w), 2256 (∪ —NCO, isocyanate, s), 1711 (∪ C═O, urethane, s), 1411 (δ C—H, Si—CH3, w), 1257 (δ C—H, siloxane, s-m), 1010 (∪ Si—O—Si, siloxane, s-m); MALDI-TOF-MS m/z: 2283 [M+Na]+ (for n=20), disubstituted

### Example 6

### Synthesis of α,ω-bis[hexyl(6-isocyanatohexyl)carbamyl]poly(dimethylsiloxane)

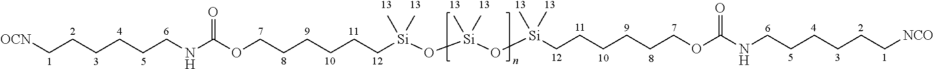

2.00 g (11.89 mmol) of 1,6-hexamethylene diisocyanate, dissolved in 20 mL of ethyl acetate, are introduced in a baked-out, argon-flushed and secured apparatus with reflux condenser and dropping funnel. The mixture is heated to 75° C. with stirring and then 0.1% by weight (16 mg) of triethylamine is added. A dropping funnel is then used to slowly add 14 g (5.8 mmol) of α,ω-bis(hydroxyhexyl)poly(dimethylsiloxane), dissolved in 30 mL of ethyl acetate, to the diisocyanate component (dropwise addition time 2 h). During this, stirring is continuous. Stirring is then performed for a further 16 h at 75° C. Yield: 15.90 g

¹H-NMR: (300 MHz, CDCl₃) δ=4.01 (m, 4H, 7), 3.27 (q, 4H, 1), 3.13 (m, 4H, 6), 1.68-1.23 (m, 32H, 2, 3, 4, 5, 8, 9, 10, 11), 0.50 (m, 4H, 12), 0.04 (m, 180H, 13) ppm.

FT-IR (diamond): \tilde{v}=2964 v(C—H), 2267 v(NCO), 1708 v(C═O)ᵤᵣₑₜₕₐₙₑ, 1523 δ(N—H), 1410 δₐₛ(C—H), 1254 δ_{sym}(C—H), 1011 v(Si—O—C), 853+789 v(SI—C) cm⁻¹.

The compounds of examples 4, 5 and 6 can then be reacted with hydroxy- or amino-functional chromophores to give the siloxane polymers according to the invention.

### Example 7

### Synthesis of isocyanate-functionalized coumarin derivatives (C-IPDI, IXa*, IXb*)

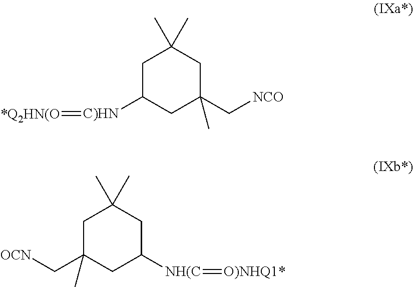

and optionally of the general formulae IXc* and/or IXd* or mixtures thereof.

### Example 8

### Synthesis of an isocyanate-functionalized coumarin derivative (C-IPDI), coumarin-IPDI, in particular synthesis of an isocyanate-functionalized umbelliferone derivative (U-IPDI) (34)

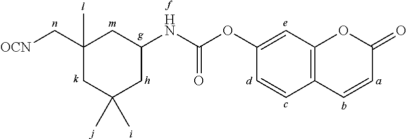

Isophorone diisocyanate (4.72 mL, 22.5 mmol) is introduced in a previously secured apparatus at RT. 1-2 drops of triethylamine are added under an argon countercurrent. A dropping funnel is then used to add dropwise 7-hydroxycoumarin (3.65 g, 22.5 mmol), which has been dissolved beforehand in a mixture of 10 mL of dichloromethane and 10 mL of dimethylformamide, to the reaction mixture over the course of 2 h. After stirring for 24 h at RT, the solvents are removed on a rotary evaporator and the product is finally dried in a high vacuum.

1H-NMR (300 MHz, CDCl3, 24° C.): δ [ppm]=7.65 (1H, d, H-b), 7.41 (1H, m, H-c), 7.08 (2H, m, H-d/H-e), 6.32 (1H, d, H-a), 3.90-3.27 (3H, m, H-g/H-n), 1.78 (2H, m, H-m), 1.28-0.78 (13H, m, H-i/H-j/H-k/H—I); FT-IR (diamond): \tilde{∪} [cm−1]=3291 (∪ N—H, urethane, w), 2927 (∪ C—H, R—CH₂—R, m-w), 2255 (∪ —NCO, isocyanate, s), 1735 (∪ C═O, urethane, s), 1660 (∪ C═O, R—CO—O—R, s), 1619 (∪ C═C, aromatic, v), 1537 (∪ C═O, urethane, m-w), 1385 (δ C—H, R—CH3, s-m) (∪ —NCO, isocyanate, m-w), 1222 (∪ C—O, R—O—Ar, s), 1152 (∪ C—O, R—O—Ar, m);

ESI-Ion-Trap-MS m/z (%): 385 [M]+

According to the synthesis as per Example 8 it is also possible to prepare a diisocyanate, in particular IPDI, with 4-amino-2,2,6,6-tetramethylpiperidine to give a piperidine isocyanate of the general formula IX as intermediate for preparing side-chain- or terminally-substituted polysiloxanes.

### Example 9

### Synthesis of a tetramethylpiperidine-terminated polydimethylsiloxane (PDMS-4-ATMP) (26)

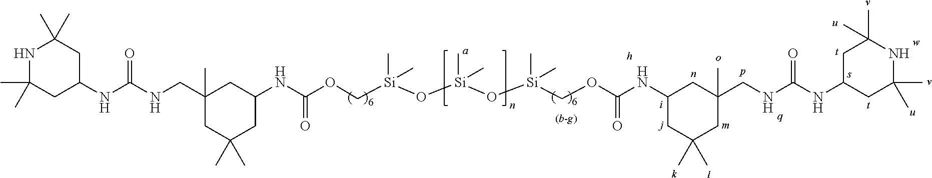

4-Amino-2,2,6,6-tetramethylpiperidine (0.60 mL, 3.5 mmol) is dissolved in 20 mL of dichlormethane in a secured round-bottomed flask together with reflux condenser and dropping funnel. Triethylamine (0.625 mL) is added. α,ω-Bis[hexyl(3-(isocyanatomethyl)-3,5,5-trimethylcyclohexyl)carbamyl]poly(dimethylsiloxane) (n=30) (4.26 g, 1.5 mmol) is likewise dissolved in 20 mL of dichloromethane and added dropwise per dropping funnel with stirring over the course of 1 h. After stirring for 24 h at RT, washing is performed with 2×20 mL of dist. water and 1×20 mL of sat. aqueous sodium hydrogencarbonate solution. The organic phases are combined, dried over sodium sulphate and the solvent is removed on a rotary evaporator. The product is then dried in high vacuum.

1H-NMR (300 MHz, CDCl3, 24° C.): δ [ppm]=3.96 (6H, m, H-g/H-s), 3.88-3.50 (2H, m, H-i), 3.24-2.74 (4H, m, H-p), 1.85 (2H, m, H-t), 1.76-1.43 (12H, m, H-f/H-n), 1.30-0.71 (66H, m, H-c/H-d/H-e H-j/H-k/H-l/H-m/H-o/H-t/H-u/H-v), 0.46 (4H, m, H-b), 0.00 (168H, m, H-a); FT-IR (diamond): \tilde{∪} [cm−1]=2961 (∪ R—CH3, Si—CH3, m-w), 2923 (∪ C—H, —CH2-, m-w), 1701 (∪ C═O, urethane, m-w), 1630 (∪ C═O, N—CO—N, v), 1559 (∪ C═O, N—CO—N, v) (6 N—H, amine, w), 1410 (δ C—H, Si—CH3, w), 1257 (δ C—H, siloxane, s-m), 1012 (∪ Si—O—Si, siloxane, s-m); MALDI-TOF-MS m/z: 1781 [M+Na]+ (for n=9), disubstituted

### Example 10

### Sensory Application Test

Testing the conditioning of hair by means of a sensory test in a hair rinse:

For the applications-related assessment of the conditioning of hair, the compounds according to the invention, Synthesis examples 2 (PDMS-umbelliferone) and 9 (PDMS-4-ATMP) and the commercially available product ABIL® Quat 3272 (INCI: Quatemium-80, manufacturer Evonik Industries) were used a a simple cosmetic hair rinse formulation.

The applications-related properties of the compounds according to the invention upon use in hair rinses were tested in the following formulations (data in % by weight based on the total formulation, made up to 100% with water):

| | Formulation examples | | | |
|---|---|---|---|---|
| | 0a | 1a | 2a | V3a |
| TEGINACID ® C, Evonik | 0.5% | 0.5% | 0.5% | 0.5% |
| Industries | | | | |
| (INCI: Ceteareth-25) | | | | |
| TEGO ® Alkanol 1618, Evonik | 5.0% | 5.0% | 5.0% | 5.0% |
| Industries (INCI: Cetearyl | | | | |
| Alcohol) | | | | |
| VARISOFT ® 300, 30% | 2.0% | 2.0% | 2.0% | 2.0% |
| strength, Evonik Industries | | | | |
| (INCI: Cetrimonium Chloride | | | | |
| (=CTAC)) | | | | |
| Neolone PE, The Dow | 0.45% | 0.45% | 0.45% | 0.45% |
| Chemical Company | | | | |
| (INCI: Phenoxyethanol; | | | | |
| Methylisothiazolinone) | | | | |
| Water, demineralized | ad 100.0% | | | |
| Citric acid | ad pH 4.5 ± 0.3 | | | |
| Synthesis example 2 | | 0.30% | | |
| (PDMS-umbelliferone) | | | | |
| Synthesis example 9 | | | 0.30% | |
| (PDMS-4-ATMP) | | | | |
| ABIL ® Quat 3272, 50% | | | | 0.60% |
| strength in propylene glycol | | | | |
| (not according to the invention) | | | | |

For the applications-related assessment, hair tresses (Kerling, Germany) were predamaged in a standardized way by a bleaching treatment. For this, customary hairstyling products were used. The test procedure, the base materials used and the details of the assessment criteria have been described in DE 103 27 871.

The hair was pretreated using a shampoo which contained no conditioners.

Standardized treatment of predamaged hair tresses with conditioning formulations:

The hair tresses, predamaged as described above, were treated as follows with the above-described conditioning rinses:

The hair tresses were wetted under running warm water (38° C., 10° dH). The excess water was gently squeezed out by hand, the shampoo is then applied and gently worked into the hair (1 ml/hair tress (2 g)). After a contact time of 1 min, the hair was rinsed for 1 min. Directly afterwards, the rinse was applied and gently worked into the hair (1 ml/hair tress (2 g)). After a contact time of 1 min, the hair was rinsed for 1 min.

Assessment Criteria:

The sensory evaluations were made according to scores which were awarded on a scale from 1 to 5, with 1 being the worst and 5 the best evaluation. The individual test criteria were each given their own evaluation.

The test criteria are: wet combability, wet feel, dry combability, dry feel, appearance/shine.

The table below compared the results of the sensory assessment of the hair tresses treated as described above with the formulations 1a and 2a according to the invention, the comparison formulation V3a and the control formulation 0a (placebo without conditioning silicone test substance).

| | Wet | Wet | Dry | Dry | |
|---|---|---|---|---|---|
| | combability | feel | combability | feel | Shine |
| Control formulation 0a | 3.9 | 3.8 | 3.9 | 4.0 | 3.0 |
| Formulation 1a | 4.5 | 4.4 | 4.5 | 4.4 | 4.0 |
| according to the | | | | | |
| invention | | | | | |
| Formulation 2a | 4.6 | 4.4 | 4.6 | 4.7 | 4.0 |
| according to the | | | | | |
| invention | | | | | |
| Comparison | 4.6 | 4.3 | 4.4 | 4.2 | 3.5 |
| formulation (not | | | | | |
| according to the | | | | | |
| invention) V3a | | | | | |

The formulations 1a and 2a according to the invention with the compounds according to the invention Example 2 and 9 exhibited good cosmetic evaluations in the sensory assessment. The control formulation 0a (with CTAC) was significantly improved by adding just 0.3% of active silicone product. Here, the already very good properties of the comparison formulation V3a, particularly with regard to dry combability and dry feel were yet further increased by the formulations 1a and 2a according to the invention. A significantly better assessment was also achieved for the shine through using the formulations 1a and 2a according to the invention. The better results compared to the comparison formulation V3a are surprising in so far as the comparison product ABIL® Quat 3272 has the same silicone chain length as Examples 2 and 9 according to the invention.

### Example 11

### Further Formulation Examples

The formulation examples given in the tables below show exemplary representatives of a large number of possible compositions according to the invention.

If the preparation of the formulation requires the separate preparation or mixing of formulation constituents beforehand, this is termed multiphase preparation.

If a two-phase preparation is required, the two phases are labelled A and B in the stated tables. In the case of three-phase processes, the three phases are called A, B and C. Unless stated otherwise, the data in the table below is data in % by weight based on the total formulation, water being used to top it up to 100%, q.s. means that as much as necessary is added and that this amount is subtracted from the amount of water:

Formulation example 1) Shampoo

| | |
|---|---|
| TEXAPON ® NSO, BASF, 28% strength | 32.00% |
| (INCI: Sodium Laureth Sulfate) | |
| Synthesis example 9 | 0.40% |
| Perfume | 0.50% |
| Water | ad 100% |
| TEGO ® Betain F 50, Evonik Industries, 38% strength | 8.00% |
| (INCI: Cocamidopropyl Betaine) | |
| ANTIL ® 171, Evonik Industries | 1.00% |
| (INCI: PEG-18 Glyceryl Oleate/Cocoate) | |
| NaCl | 0.50% |
| Preservative | q.s. |

Formulation example 2) Shampoo, PEG- & sulphate-free

| | |
|---|---|
| REWOTERIC ® AM C, Evonik Industries, 32% strength | 15.00% |
| (INCI: Sodium Cocoamphoacetate) | |
| Plantapon ACG 50, BASF | 3.80% |
| (INCI: Disodium Cocoyl Glutamate) | |
| Synthesis example 9 | 1.00% |
| Perfume | 0.30% |
| Water | ad 100% |
| TEGO ® Betain F 50, Evonik Industries, 38% strength | 10.00% |
| (INCI: Cocamidopropyl Betaine) | |
| VARISOFT ® PATC, Evonik Industries | 2.30% |
| (INCI: Palmitamidopropyltrimonium Chloride) | |
| ANTIL ® SPA 80, Evonik Industries | 2.00% |
| (INCI: Isostearamide MIPA; Glyceryl Laurate) | |
| Preservative | 0.30% |
| Citric Acid, 30% strength | q.s. |

Formulation example 3) Conditioning Shampoo

| | |
|---|---|
| TEXAPON ® NSO, BASF, 28% strength | 32.00% |
| (INCI: Sodium Laureth Sulfate) | |
| ANTIL ® 200, Evonik Industries (INCI: PEG-200 | 2.00% |
| Hydrogenated Glyceryl Palmate; PEG-7 Glyceryl | |
| Cocoate) | |
| Synthesis example 9 | 0.50% |
| Perfume | 0.25% |
| Water | ad 100% |
| Polymer JR 400, Amerchol | 0.20% |
| (INCI: Polyquaternium-10) | |
| TEGO ® Betain F 50, Evonik Industries, 38% strength | 8.00% |
| (INCI: Cocamidopropyl Betaine) | |
| NaCl | 0.30% |
| Preservative | q.s. |

Formulation example 4) Conditioning Shampoo

| | |
|---|---|
| TEXAPON ® NSO, BASF, 28% strength | 32.00% |
| (INCI: Sodium Laureth Sulfate) | |
| ANTIL ® 200, Evonik Industries (INCI: PEG-200 | 2.00% |
| Hydrogenated Glyceryl Palmate; PEG-7 Glyceryl | |
| Cocoate) | |
| ABIL ® Quat 3272, Evonik Industries (INCI: | 0.75% |
| Quaternium-80) | |
| Synthesis example 9 | 1.50% |
| Synthesis example 2 | 0.40% |
| Perfume | 0.25% |
| Water | ad 100% |
| Jaguar C-162, Rhodia | 0.10% |
| (INCI: Hydroxypropyl Guar Hydroxypropyltrimonium | |
| Chloride) | |
| Polymer JR 400, Amerchol | 0.20% |
| (INCI: Polyquaternium-10) | |
| REWOTERIC ® AM C, Evonik Industries, 32% strength | 3.00% |
| (INCI: Sodium Cocoamphoacetate) | |
| TEGO ® Betain F 50, Evonik Industries, 38% strength | 5.00% |
| (INCI: Cocamidopropyl Betaine) | |
| TEGO ® Pearl N 300 Evonik Industries | 2.00% |
| (INCI: Glycol Distearate; Laureth-4; Cocamidopropyl | |
| Betaine) | |
| NaCl | 0.30% |
| Preservative | q.s. |

Formulation example 5) Shampoo, PEG- & sulphate-free

| | | |
|---|---|---|
| A | REWOTERIC ® AM C, Evonik Industries, 32% | 20.00% |
| | strength | |
| | (INCI: Sodium Cocoamphoacetate) | |
| | REWOPOL ® SB F 12 P, Evonik Goldschmidt, 96% | 5.90% |
| | strength | |
| | (INCI: Disodium Lauryl Sulfosuccinate) | |
| | Synthesis example 2 | 1.00% |
| | ANTIL ® SPA 80, Evonik Industries, | 1.70% |
| | (INCI: Isostearamide MIPA; Glyceryl Laurate) | |
| B | Water | ad 100% |
| | Citric Acid, 30% strength | 3.60% |
| C | ANTIL ® HS 60, Evonik Industries, | 3.00% |
| | (INCI: Cocamidopropyl Betaine; Glyceryl Laurate) | |
| | Preservative | 0.60% |

Formulation example 6) Rinse-Off Conditioner

| | |
|---|---|
| Water | ad 100% |
| VARISOFT ® EQ 65, Evonik Industries | 2.00% |
| (INCI: Distearoylethyl Dimonium Chloride; Cetearyl | |
| Alcohol) | |
| VARISOFT ® BT 85, Evonik Industries | 1.00% |
| (INCI: Behentrimonium Chloride) | |
| Synthesis example 9 | 1.10% |
| TEGO ® Alkanol 1618, Evonik Industries | 5.00% |
| (INCI: Cetearyl Alcohol) | |
| Preservative, Perfume | q.s. |

Formulation example 7) Rinse-Off Conditioner

| | |
|---|---|
| Water | ad 100% |
| VARISOFT ® EQ 65, Evonik Industries | 2.00% |
| (INCI: Distearoylethyl Dimonium Chloride; | |
| Cetearyl Alcohol) | |
| VARISOFT ® BT 85, Evonik Industries | 2.00% |
| (INCI: Behentrimonium Chloride) | |
| ABIL ® Quat 3272, Evonik Industries | 0.50% |
| (INCI: Quaternium-80) | |
| Synthesis example 2 | 1.30% |
| Synthesis example 9 | 0.50% |
| TEGO ® Alkanol 1618, Evonik Industries | 5.00% |
| (INCI: Cetearyl Alcohol) | |
| Preservative, Perfume | q.s. |

Formulation example 8) Rinse-Off Conditioner

| | |
|---|---|
| TEGINACID ® C, Evonik Industries (INCI: Ceteareth-25) | 0.50% |
| TEGO ® Alkanol 16, Evonik Industries (INCI: Cetyl | 2.00% |
| Alcohol) | |
| TEGO ® Amid S 18, Evonik Industries | 1.00% |
| (INCI: Stearamidopropyl Dimethylamine) | |
| Synthesis example 2 | 0.50% |
| Propylene Glycol | 2.00% |
| Citric Acid Monohydrate | 0.30% |
| Water | ad 100% |
| Preservative, Perfume | q.s. |

Formulation example 9) Leave-In Conditioner Spray

| | |
|---|---|
| Lactic Acid, 80% | 0.40% |
| Water | ad 100% |
| TEGO ® Amid S 18, Evonik Industries | 1.20% |
| (INCI: Stearamidopropyl Dimethylamine) | |
| TEGIN ® G 1100 Pellets, Evonik Industries | 0.60% |
| (INCI: Glycol Distearate) | |
| TEGO ® Care PS, Evonik Industries | 1.20% |
| (INCI: Methyl Glucose Sesquistearate) | |
| TEGOSOFT ® DEC, Evonik Industries | 0.30% |
| (INCI: Diethylhexyl Carbonate) | |
| Synthesis example 9 | 1.20% |
| Preservative, Perfume | q.s. |

Formulation example 10) Leave-In Conditioner Spray

| | |
|---|---|
| TAGAT ® CH 40, Evonik Industries | 6.00% |
| (INCI: PEG-40 Hydrogenated Castor Oil) | |
| Ceramide VI, Evonik Industries (INCI: | 0.05% |
| Ceramide 6 II) | |
| Perfume | 0.20% |
| Water | ad 100% |
| Synthesis example 2 | 2.00% |
| LACTIL ®, Evonik Industries | 2.00% |
| (INCI: Sodium Lactate; Sodium PCA; Glycine; | |
| Fructose; Urea; Niacinamide; Inositol; Sodium | |
| benzoate; Lactic Acid) | |
| TEGO ® Betain F 50, Evonik Industries | 4.30% |
| 38% (INCI: Cocamidopropyl Betaine) | |
| Citric Acid (10% in water) | 2.00% |

Formulation example 11) Leave-In Conditioner Foam

| | |
|---|---|
| Synthesis example 2 | 0.50% |
| TAGAT ® CH 40, Evonik Industries | 0.90% |
| (INCI: PEG-40 Hydrogenated Castor Oil) | |
| Perfume | 0.30% |
| TEGO ® Betain 810, Evonik Industries | 2.00% |
| (INCI: Capryl/Capramidopropyl Betaine) | |
| Water | ad 100% |
| TEGO ® Cosmo C 100, Evonik Industries (INCI: | 0.50% |
| Creatine) | |
| TEGOCEL ® HPM 50, Evonik Industries | 0.30% |
| (INCI: Hydroxypropyl Methylcellulose) | |
| VARISOFT ® 300, Evonik Industries | 1.30% |
| (INCI: Cetrimonium Chloride) | |
| LACTIL ® Evonik Industries | 0.50% |
| (INCI: Sodium Lactate; Sodium PCA; Glycine; | |
| Fructose; Urea; Niacinamide; Inositol; Sodium | |
| benzoate; Lactic Acid) | |
| Citric Acid (30% in water) | 0.10% |
| Preservative | q.s. |

Formulation example 12) Strong Hold Styling Gel PGP-5,T1

| | |
|---|---|
| TEGO ® Carbomer 141, Evonik Industries (INCI: | 1.20% |
| Carbomer) | |
| Water | ad 100% |
| NaOH, 25% | 2.70% |
| PVP/VA W-735, ISP | 16.00% |
| (INCI: PVP/VA Copolymer) | |
| Synthesis example 2 | 0.50% |
| Synthesis example 9 | 1.00% |
| Alcohol Denat. | 10.00% |
| TAGAT ® O 2 V, Evonik Industries | 2.00% |
| (INCI: PEG-20 Glyceryl Oleate) | |
| Perfume | 0.30% |
| ABIL ® B 88183, Evonik Industries | 0.30% |
| (INCI: PEG/PPG-20/6 Dimethicone) | |
| Preservative | q.s. |

Formulation example 13) Foaming body care composition

| | |
|---|---|
| TEXAPON ® NSO, BASF, 28% strength | 14.30% |
| (INCI: Sodium Laureth Sulfate) | |
| Perfume | 0.30% |
| Synthesis example 2 | 1.00% |
| REWOTERIC ® AM C, Evonik Industries, 32% strength | 8.00% |
| (INCI: Sodium Cocoamphoacetate) | |
| Water | ad 100% |
| Polyquaternium-7, Nalco, (INCI: Merquat 550) | 0.30% |
| LACTIL ®, Evonik Industries | 0.50% |
| (INCI: Sodium Lactate; Sodium PCA; Glycine; | |
| Fructose; Urea; Niacinamide; Inositol; Sodium | |
| benzoate; Lactic Acid) | |
| Citric Acid Monohydrate | 0.50% |

Formulation example 14) Mild Foam Bath

| | |
|---|---|
| TEXAPON ® NSO, BASF, 28% strength | 27.00% |
| (INCI: Sodium Laureth Sulfate) | |
| REWOPOL ® SB FA 30, Evonik Industries, 40% | 12.00% |
| strength | |
| (INCI: Disodium Laureth Sulfosuccinate) | |
| TEGOSOFT ® LSE 65 K SOFT, Evonik Industries | 2.00% |
| (INCI: Sucrose Cocoate) | |
| Water | ad 100% |
| REWOTERIC ® AM C, Evonik Industries, 32% strength | 13.00% |
| (INCI: Sodium Cocoamphoacetate) | |
| Synthesis example 9 | 0.30% |
| Citric Acid (30% in water) | 3.00% |
| ANTIL ® 171, Evonik Industries | 1.50% |
| (INCI: PEG-18 Glyceryl Oleate/Cocoate) | |
| TEGO ® Pearl N 300 Evonik Industries | 2.00% |
| (INCI: Glycol Distearate; Laureth-4; Cocamidopropyl | |
| Betaine) | |

Formulation example 15) Rinse-Off Conditioner

| | |
|---|---|
| Water | ad 100% |
| VARISOFT ® 300, Evonik Industries | 2.00% |
| (INCI: Cetrimonium Chloride) | |
| VARISOFT ® BT 85, Evonik Industries | 2.00% |
| (INCI: Behentrimonium Chloride) | |
| ABIL ® OSW 5, Evonik Industries | 1.00% |
| (INCI: Cyclopentasiloxane; Dimethiconol) | |
| Synthesis example 2 | 0.80% |
| TEGO ® Alkanol 1618, Evonik Industries | 5.00% |
| (INCI: Cetearyl Alcohol) | |
| Preservative, Perfume | q.s. |

Formulation example 16) Rinse-Off Conditioner

| | |
|---|---|
| Water | ad 100% |
| VARISOFT ® BT 85, Evonik Industries | 3.00% |
| (INCI: Behentrimonium Chloride) | |
| SF 1708, Momentive | 2.00% |
| (INCI: Amodimethicone) | |
| Synthesis example 9 | 0.50% |
| Synthesis example 2 | 0.80% |
| TEGO ® Alkanol 1618, Evonik Industries | 5.00% |
| (INCI: Cetearyl Alcohol) | |
| Preservative, Perfume | q.s. |

Formulation example 17, moisturizing skin cleanser

| | | |
|---|---|---|
| A | TEXAPON ® NSO, BASF, 28% strength | 30.00% |
| | (INCI: Sodium Laureth Sulfate) | |
| | Synthesis example 2 | 0.70% |
| | Perfume | 0.30% |
| B | Water | ad 100% |
| | TEGOCEL ® fluid HPM 4000, Evonik Industries | 1.20% |
| | (INCI: Hydroxypropyl Methylcellulose) | |
| | TEGO ® Betain C 60, Evonik Industries, 46% | 8.10% |
| | strength | |
| | (INCI: Cocamidopropyl Betaine) | |
| | TEGOSOFT ® APM, Evonik Industries | 1.00% |
| | (INCI: PPG-3 Myristyl Ether) | |
| | Cutina TS, BASF (INCI: PEG-3 Distearate) | 1.00% |
| | REWODERM ® LI S 80, Evonik Industries | 1.50% |
| | (INCI: PEG-200 Hydrogenated Glyceryl Palmate; | |
| | PEG-7 Glyceryl Cocoate) | |
| | Preservative | 0.60% |
| | Citric Acid, 30% strength | q.s. |

Formulation example 18, Turbid Conditioning Shampoo

| | |
|---|---|
| TEXAPON ® NSO, BASF, 28% strength | 32.00% |
| (INCI: Sodium Laureth Sulfate) | |
| ANTIL ® 200, Evonik Industries (INCI: PEG-200 | 2.00% |
| Hydrogenated Glyceryl Palmate; PEG-7 Glyceryl | |
| Cocoate) | |
| Synthesis example 2 | 1.00% |
| Perfume | 0.25% |
| Water | ad 100% |
| Polymer JR 400, Amerchol (INCI: Polyquaternium-10) | 0.20% |
| TEGO ® Betain F 50, Evonik Industries, 38% strength | 8.00% |
| (INCI: Cocamidopropyl Betaine) | |
| DC1503 Fluid, Dow Corning | 1.00% |
| (INCI: Dimethicone; Dimethiconol) | |
| TEGO ® Pearl N 300 Evonik Industries | 2.00% |
| (INCI: Glycol Distearate; Laureth-4; Cocamidopropyl | |
| Betaine) | |
| NaCl | 0.30% |
| Preservative | q.s. |

Formulation example 19) Mild Hair & Body Wash, PEG- and Sulphate-free

| | |
|---|---|
| Plantacare ® 1200 UP, BASF, 50% strength | 11.40% |
| (INCI: Lauryl Glucoside) | |
| Plantacare ® 818 UP, BASF, 51% | 5.60% |
| (INCI: Coco Glucoside) | |
| Water | ad 100% |
| ANTIL ® Soft SC, Evonik Industries | 0.90% |
| (INCI: Sorbitan Sesquicaprylate) | |
| Synthesis example 2 | 1.00% |
| TEGOSOFT ® LSE 65 K SOFT, Evonik Industries | 1.50% |
| (INCI: Sucrose Cocoate) | |
| TEGO ® Betain F 50, Evonik Industries, 38% strength | 18.00% |
| (INCI: Cocamidopropyl Betaine) | |
| Perfume, preservative | q.s. |
| Citric Acid, 30% | q.s. |

Formulation example 20) Sprayable Hairmilk, PEG-free

| | | |
|---|---|---|
| A | Water | ad 100% |
| | Lactic Acid, 80% strength | 0.40% |
| B | TEGO ® AMID S 18, Evonik Industries | 1.20% |
| | (INCI: Stearamidopropyl Dimethylamine) | |
| | TEGIN ® G 1100 Pellets, Evonik Industries | 0.60% |
| | (INCI: Glycol Distearate) | |
| | TEGO ® Care PS, Evonik Industries | 1.20% |
| | (INCI: Methyl Glucose Sesquistearate) | |
| | TEGOSOFT ® DEC, Evonik Industries | 0.30% |
| | (INCI: Diethylhexyl Carbonate) | |
| | Synthesis example 2 | 0.60% |
| | Perfume, preservative | q.s. |

Formulation example 21: Conditioning Anti-dandruff Shampoo

| | | |
|---|---|---|
| A | TEGIN ® G 1100 Pellets, Evonik Industries | 3.00% |
| | (INCI: Glycol Distearate) | |
| | TEXAPON ® NSO, Cognis, 28% strength | 40.00% |
| | (INCI: Sodium Laureth Sulfate) | |
| B | Perfume | 0.30% |
| | Zinc-Pyrion NF, WeylChem, 48% strength | 2.00% |
| | (INCI: Zinc Pyrithione) | |
| | Synthesis example 9 | 1.00% |
| C | Water | ad 100% |
| | TEGO ® Carbomer 341 ER, Evonik Industries | 0.20% |
| | (INCI: Acrylates/C10-30 Alkyl Acrylate | |
| | Crosspolymer) | |
| | Water | 0.30% |
| | NaOH, 25% strength | 0.30% |
| D | REWOTERIC ® AM B U 185, Evonik Industries, | 12.50% |
| | 30% strength | |
| | (INCI: Undecylenamidopropyl Betaine) | |
| | ANTIL ® SPA 80, Evonik Industries | 3.70% |
| | (INCI: Isostearamide MIPA; Glyceryl Laurate) | |
| E | Preservative | q.s. |

Formulation example 22: Hair colorant

| Water demineralized | ad 100% |
|---|---|
| TEGO ® Alkanol 1618, Evonik Industries, | 12.00% |
| (INCI: Cetearyl Alcohol) | |
| Eutanol ® G, BASF (INCI: Octyldodecanol) | 3.00% |
| REWOMID ® C 212, Evonik Industries | 1.50% |
| (INCI: Cocamide MEA) | |
| Super Hartolan ® B, Crodo (INCI: Lanolin Alcohol) | 3.00% |
| Avocado oil, Henry Lamotte (INCI: Persea Gratissima Oil) | 1.50% |
| Pristerene ® 4960, Uniquema (INCI: Stearic Acid) | 6.00% |
| EDTA BD, BASF (INCI: Disodium EDTA) | 0.10% |
| Texapon ® K12G, BASF (INCI: Sodium Lauryl Sulfate) | 0.50% |
| Propylene glycol | 5.00% |
| Timica Silver Sparkle, BASF (INCI: MICA; Titanium | 1.00% |
| Dioxide) | |
| Ammonia solution, 25% strength | 6.00% |
| 2,5-Diaminotoluene sulphate, (INCI: Toluene-2,5-Diamine) | 1.40% |
| Rodol ® RS, Jos. H. Lowenstein & Sons (INCI: Resorcinol) | 0.30% |
| HC Blue A42, (INCI: 2,4-Diaminophenoxyethanol di HCl) | 0.10% |
| Sodium sulphite | 0.50% |
| Perfume | 0.20% |
| Synthesis example 9 | 0.50% |

Formulation example 23: Shampoo

| | |
|---|---|
| TEXAPON ® NSO, Cognis, 28% strength | 28.00% |
| (INCI: Sodium Laureth Sulfate) | |
| REWOTERIC ® AM 2 C NM, Evonik Industries, 39% | 4.00% |
| strength (INCI: Disodium Cocoamphodiacetate) | |
| TEGO ® Betain F 50, Evonik Industries, 38% strength | 7.00% |
| (INCI: Cocamidopropyl Betaine) | |
| REWOMID ® C 212, Evonik Industries | 0.80% |
| (INCI: Cocamide MEA) | |
| ANTIL ® 171, Evonik Industries | 0.50% |
| (INCI: PEG-18 Glyceryl Oleate/Cocoate) | |
| N-Hance ® SP-100, Hercules | |
| (INCI: Acrylamidopropyl Trimonium Chloride/Acrylamide | |
| Copolymer) | |
| Polymer JR 400, Amerchol | 0.10% |
| (INCI: Polyquaternium-10) | |
| Jaguar C-162, Rhodia | 0.20% |
| (INCI: Hydroxypropyl Guar Hydroxypropyltrimonium | |
| Chloride) | |
| DC 193, Dow Corning | 0.40% |
| (INCI: PEG-12 Dimethicone) | |
| Synthesis example 2 | 0.70% |
| Synthesis example 9 | 0.60% |
| TEGIN ® D 1102, Evonik Industries | 0.40% |
| (INCI: PEG-3 Distearate) | |
| TAGAT ® CH 40, Evonik Industries | 0.20% |
| (INCI: PEG-40 Hydrogenated Castor Oil) | |
| Water | ad 100% |
| NaCl | 0.70% |
| Citric Acid | ad |
| | pH = ~5.5 |
| Perfume | q.s. |
| Preservative | q.s. |

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. A siloxane polymer of the general formula I comprising a central polysiloxane polymer block B,
(i.) which is substituted with organofunctional radicals,
(ii.) the polymer block B has linear and/or branched structures with at least two difunctional siloxane units,
(iii.) the polymer block B has on at least two terminal silicon atoms or at least one terminal and at least one lateral silicon atom of the siloxane units of polymer block B the organofunctional radicals -Q1 and -Q2, where radicals are identical or different,
Q2-B-Q1 (I)
where -Q1 corresponds to the general formula IIa and -Q2 corresponds to the formula IIb,
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
where A is —NH—, —O— or —S— and D is —NH— in each case independently in formulae IIa and IIb,
where A′ is —NH— and D′ is —NH—, —O— or —S— in each case independently in formulae IIa and IIb, where each radical Q1 and Q2 of the formulae IIa or IIb in each case independently has at least two bivalent groups selected from carbamate and urea group,
where Q1′ and Q2′ in each case independently comprises a bivalent hydrocarbon radical with 6 to 200 carbon atoms optionally comprising
at least one heteroatom selected from O, N and S, a bivalent radical
comprising aryl, arylalkyl groups optionally comprising polyether radicals containing at least one heteroatom O, N or S or polyether radicals containing alkyl, aryl or alkyl and aryl groups,
where Q1″ and Q2″ in each case independently comprises a bivalent linear,
branched and/or cyclic alkyl radical with 4 to 200 carbon atoms or a
bivalent radical comprising an aryl and/or arylalkyl radical with 6 to 200
carbon atoms, and
where -D′-Q1*
and -D′-Q2* in each case independently comprises as radicals Q1* and Q2* a UV/Vis chromophore as radical.

2. The siloxane polymer according to claim 1, wherein
the siloxane polymer of the general formulae I, IIa and IIb, -D′-Q1* and
-D′-Q2* in each case independently comprises as radicals a UV chromophore with at least one absorption maximum in the range from 280 to 380 nm.

3. The siloxane polymer according to claim 1, wherein
the siloxane polymer of the general formulae I, IIa and IIb, -D′-Q1* and
-D′-Q2* in each case independently comprises a Vis chromophore as radical with at least one absorption maximum in the range from 320 to 790 nm.

4. The siloxane polymer according to claim 1, wherein
in the siloxane polymer of the general formula I, the polymer block B corresponds to the general formula IIIa or IIIb, where B is 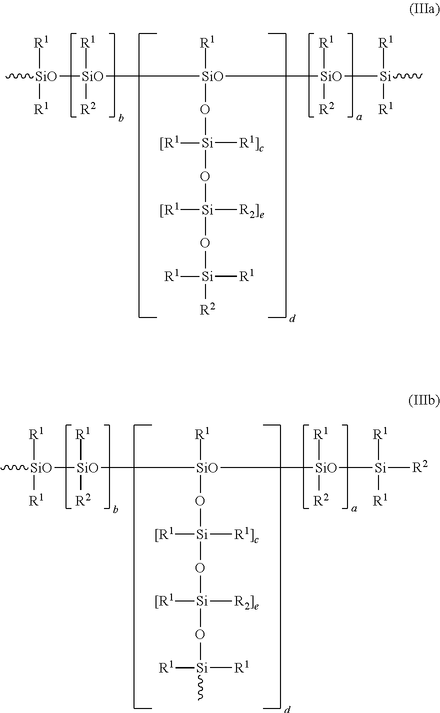
where a, b, c, d and e in formulae IIIa and IIIb are in each case independently an integer
where a is from 1 to 200,
where b is from 0 to 200,
where c is from 0 to 200,
where d is from 0 to 200,
where e is from 0 to 200 and
where R¹ in formulae IIIa or IIIb in each case independently are identical or different,
where R¹ comprises alkyl radicals having 1 to 22 carbon atoms, or phenyl radicals,
where R² in formulae IIIa or IIIb is alkyl radical having 1 to 22 carbon atoms, an alkyl radical with at least one heteroatom selected from N, O, S or phenyl radical.

5. The siloxane polymer according to claim 1, wherein
b, c, d and e are 0 and a is 20 to 100.

6. The siloxane polymer according to claim 1, wherein
R¹ and R² are selected from alkyl groups with 1, 2, 3 or 4 carbon atoms.

7. The siloxane polymer according to claim 1, wherein
the radicals -Q1 and -Q2 in the general formula I are selected independently from
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
a) where A is —O—, D is —NH—, A′ is —NH— and D′ is —O—,
b) where A is —O—, D is —NH—, A′ is —NH— and D′ is —NH—,
c) where A is —NH—, D is —NH—, A′ is —NH— and D′ is —NH—,
d) where A is —S—, D is —NH—, A′ is —NH— and D′ is —NH—,
e) where A is —NH—, D is —NH—, A′ is —NH— and D′ is —O— or
f) where A is —S—, D is —NH—, A′ is —NH— and D′ is —O—,
g) where A is —O—, D is —NH—, A′ is —NH— and D′ is —S—,
h) where A is —NH—, D is —NH—, A′ is —NH— and D′ is —S— or
i) where A is —S—, D is —NH—, where A′ is —NH— and D′ is —S—.

8. The siloxane polymer according to claim 1, wherein
in the radicals -Q1 and -Q2 of the formula I, the radicals -D′-Q1* and -D′-Q2* are in each case independently derived from a hydroxycoumarin or hydroxycoumarin derivative, or an amino-coumarin or amino-coumarin derivative.

9. The siloxane polymer according to claim 1, wherein
in the radicals -Q1 and -Q2 of the formula I,
A is —O—, D is —NH—, A′ is —NH— and D′ is —O—, and the radicals
-D′-Q1* and -D′-Q2* are in each case independently selected from a hydroxycoumarin or hydroxycoumarin derivative.

10. The siloxane polymer according to claim 1, wherein
in the siloxane polymer of the general formulae I, IIa and IIb, the radicals are selected from -D′-Q1* and -D′-Q2* in each case independently where D′ is —NH— and where Q1* or Q2* is an aminofunctional hydrocarbon radical.

11. The siloxane polymer according to claim 1, wherein
in the radicals -Q1 and -Q2 of the formula I, the bivalent radicals -Q1″- and -Q2″- are selected independently from bivalent, linear, branched or cyclic alkylene radicals with 4 to 25 carbon atoms.

12. The siloxane polymer according to claim 1, wherein
in the radicals -Q1 and -Q2 of the formula I, the bivalent radicals -Q1′- and -Q2′-are selected from alkylene radicals with 3 bis 22 carbon atoms optionally with at least one heteroatom comprising N, O or S or from polyether radicals containing alkyl, aryl or alkyl and aryl groups of the formulae IVa or IVb
where Q1′ and Q2′ are in each case independently
-T-O—(CH₂—CH₂—O—)ₓ—(CH₂—CH(R^{#})O—)_{y}—(SO)—R″ (IVa)
-T-O—(CH₂—CH₂—O—)ₓ—(CH₂—CH(R^{#})O—)_{y}—R″ (IVb),
where T=bivalent hydrocarbon radical with 2 to 4 carbon atoms,
where x=0 to 200,
y=0 to 200,
where x and y are integers, with the proviso that x or y is at least 1, where R^{#} is hydrogen or methyl,
where R″ is alkylene.

13. The siloxane polymer according to claim 1, wherein
in the radicals -Q1 and -Q2 of the formula I, at least one of the bivalent radicals -Q1″- and -Q2″- is independently a bivalent cyclohexane-containing radical selected from the formulae Va and Vb

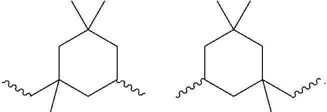

14. The siloxane polymer according to claim 1, wherein
the siloxane of the general formula I corresponds to the siloxane polymer of the general formula XI where
Mₐ₁M^{A}ₐ₂M^{B}ₐ₃D_{b1}D^{A}_{b2}D^{B}_{b3}T_{c1}T^{A}c2T^{B}_{c3}Q_{d1} (XI)
where M=[R¹⁶₃SiO_{1/2}], M^{A}=[R¹⁷R¹⁶₂SiO_{1/2}], M^{B}=[R¹⁸R¹⁶₂SiO_{1/2}], D=[R¹⁶₂SiO_{2/2}], D^{A}=[R¹⁷₁R¹⁶₁SiO_{2/2}], D^{B}=[R¹⁸R¹⁶₁SiO_{2/2}], T=[R¹⁶SiO_{3/2}], T^{A}=[R¹⁷SiO_{3/2}], T^{B}=[R¹⁸SiO_{3/2}], Q=[SiO_{4/2}],
where R¹⁶ is independently of one another identical or different linear or branched, saturated or unsaturated hydrocarbon radicals with 1 to 30 carbon atoms or else aromatic hydrocarbon radicals with 6 to 30 carbon atoms,
where R¹⁷ is in each case independently -Q1 or -Q2,
where R¹⁸ is independently of one another identical or different linear or branched, saturated or olefinically unsaturated hydrocarbon radicals with 8 to 30 carbon atoms, aromatic hydrocarbon radical with 6 to 40 carbon atoms, alkylaryl radical with 7 to 40 carbon atoms, a linear or branched optionally double-bond-containing aliphatic hydrocarbon radical with 2 to 30 carbon atoms that is interrupted by one or more heteroatoms, such as oxygen, NH, NR′ where R′ is an optionally double-bond-containing C1 to C30 alkyl radical, a linear or branched optionally double-bond-containing aliphatic hydrocarbon radical with 2 to 30 carbon atoms interrupted by one or more functionalities selected from the group —OH, —O—C(O)—, —(O)C—O—, —NH—C(O)—, —(O)C—NH, —(CH₃)N—C(O)—, —(O)C—N(CH₃)—, —S(O₂)—O—, —O—S(O₂)—, —S(O₂)—NH—, —NH—S(O₂)—, —S(O₂)—N(CH₃)—, —N(CH₃)—S(O₂)—, a linear or branched, optionally double-bond-containing aliphatic or cycloaliphatic hydrocarbon radical with 1 to 30 carbon atoms functionalized terminally with OH, OR′, NH², N(H)R′, N(R′)₂ where R′ is an optionally double-bond containing C1- to C30-alkyl radical, or a blockwise or randomly structured polyether according to —(R⁵—O)ₙ—R⁶, where R⁵ is a linear or branched hydrocarbon radical containing 2 to 4 carbon atoms, n is 1 to 100, and R⁶ is hydrogen, a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms, or a radical —C(O)—R⁷ where R⁷ is a linear or branched optionally double-bond-containing aliphatic hydrocarbon radical with 1 to 30 carbon atoms, an optionally double-bond-containing cycloaliphatic hydrocarbon radical with 5 to 40 carbon atoms, an aromatic hydrocarbon radical with 6 to 40 carbon atoms, an alkylaryl radical with 7 to 40 carbon atoms,
with the indices
a1=0-200,
a2=0-30,
a3=0-30,
b1=2 to 5000,
b2=0 to 100,
b3=0 to 100, c1=0 to 30,
c2=0 to 30, c3=0 to 30,
d1=0 to 30,
with the proviso that at least one of the indices selected from a2 and a3 is not 0,

15. The siloxane polymer according to claim 1,
wherein the siloxane polymer is selected from siloxane polymers of the formulae Ia and Ib or mixtures of these 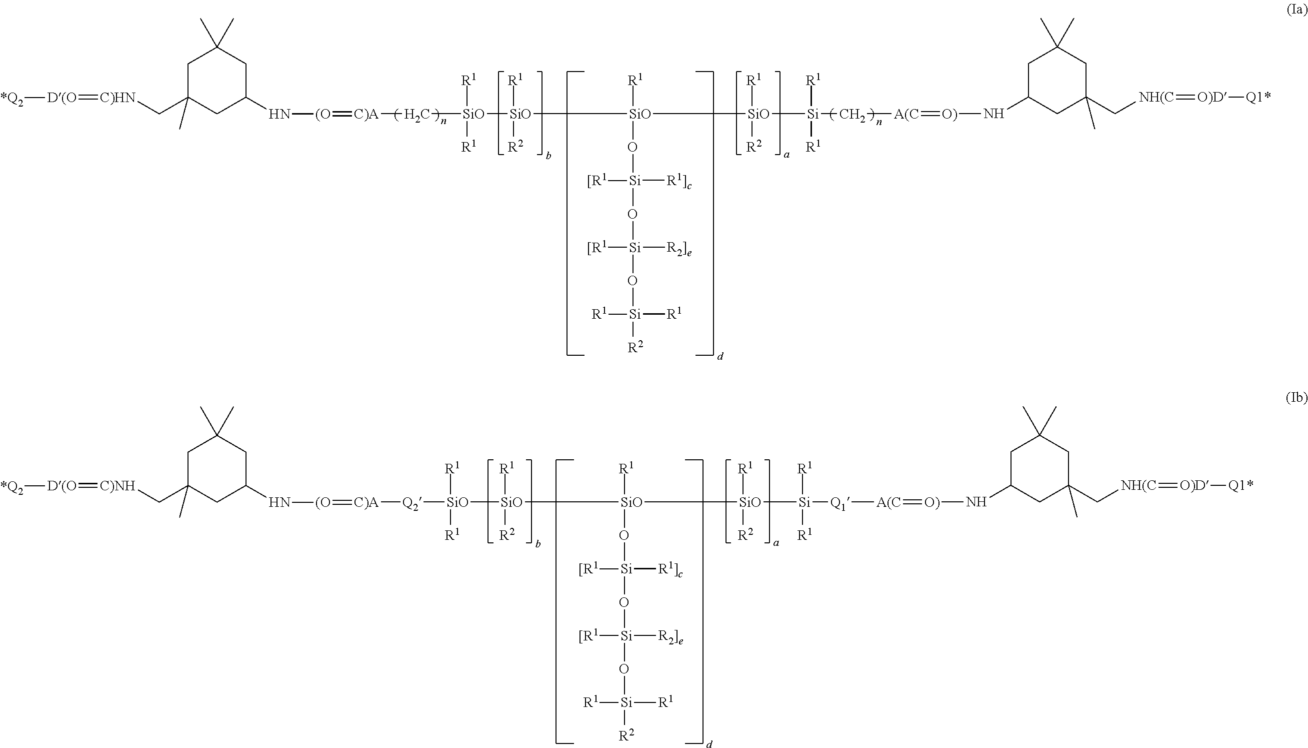
where n or n′ is in each case independently selected from an integer from 3 to 22,
where a is from 1 to 200,
where b is from 0 to 200,
where c is from 0 to 200,
where d is from 0 to 200,
where e is from 0 to 200 and
where R¹ in formulae Ia and Ib are in each case independently identical or different,
where R¹ comprises alkyl radicals with 1 to 4 carbon atoms or phenyl radicals,
where R² is alkyl radical with 1 to 22 carbon atoms, an alkyl radical with at least one heteroatom selected from N, O, S, or phenyl radical, and
where -D′-Q1* and -D′-Q2* are in each case independently derived from a hydroxy-coumarin or hydroxy-coumarin derivative, and
where Q1′ and Q2′ in formula Ib are in each case independently alkylene with 2 to 40 carbon atoms or a polyether of the formulae IVa or IVb
-T-O—(CH₂—CH₂—O—)ₓ—(CH₂—CH(R^{#})O—)_{y}—(SO)—R″ (IVa)
-T-O—(CH₂—CH₂—O—)ₓ—(CH₂—CH(R^{#})O—)_{y}—R″ (IVb)
where T=bivalent hydrocarbon radical with 2 to 4 carbon atoms,
where x=0 to 200,
y=0 to 200,
where x and y are integers with the proviso that x or y is at least 1,
where R^{#} is hydrogen or methyl, and
where R″ is alkylene, in particular ethylene.

16. The siloxane polymer according to claim 1, wherein
the siloxane polymer is selected from siloxane polymers of the formula Ia* and Ib* 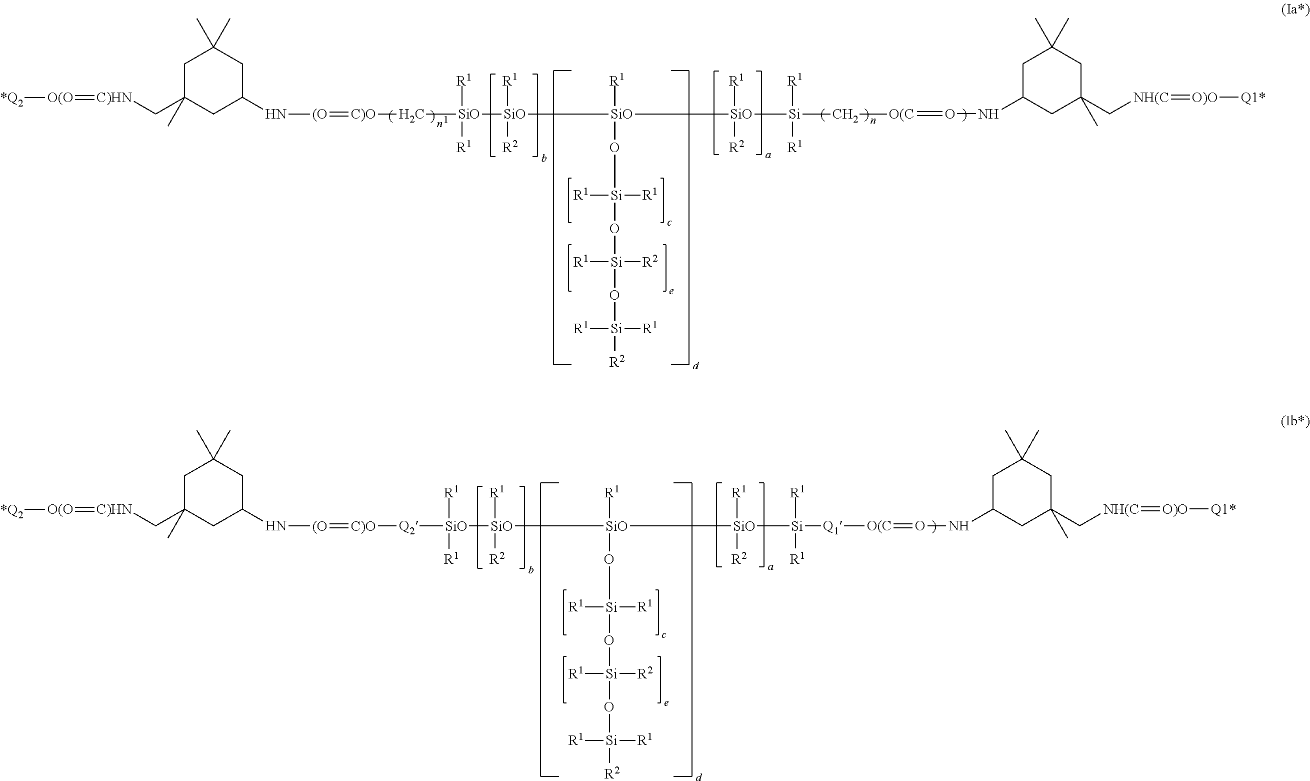
where n or n′ is in each case independently selected from an integer from 3 to 22,
where a is from 1 to 200,
where b is from 0 to 200,
where c is from 0 to 200,
where d is from 0 to 200,
where e is from 0 to 200 and
where R¹ in formulae Ia* and Ib* are in each case independently identical or different,
where R¹ includes alkyl radicals with 1 to 4 carbon atoms or phenyl radicals,
where R² is alkyl radical with 1 to 22 carbon atoms, an alkyl radical with at least one heteroatom comprising N, O, S where n or n′ is in each case independently selected from an integer from 3 to 22 or phenyl radical, and
where Q1* and Q2* are in each case independently coumarin or a coumarin derivative and in formula Ib* where Q1′ and Q2′ are in each case independently
-T-O—(CH₂—CH₂—O—)ₓ—(CH₂—CH(R^{#})O—)_{y}—(SO)—R″ (IVa)
-T-O—(CH₂—CH₂—O—)ₓ—(CH₂—CH(R^{#})O—)_{y}—R″ (IVb),
where T=bivalent hydrocarbon radical with 2 to 4 carbon atoms,
where x=0 to 200,
y=0 to 200,
where x and y are integers with the proviso that x or y is at least 1,
where R^{#} is hydrogen or methyl, and
where R″ is hydrogen or alkylene.

17. A process for the preparation of a siloxane polymer and of compositions comprising these siloxane polymers or mixtures of the siloxane polymers with a central polysiloxane polymer block B, by
a) reacting a polysiloxane diisocyanate of the formula VII with at least one hydroxy- or amino-functional UV/Vis chromophore, giving a siloxane polymer of the general formula I
OCN-Q2″-D-(O═C)-A-Q2′-B-Q1′-A-(C═O)-D-Q1″-NCO (VII)
Q2-B-Q1 (I)
where -Q1 corresponds to the general formula IIa and -Q2 corresponds to the formula IIb,
-Q1=-Q1′-A-(C═O)-D-Q1″-A′-(C═O)-D′-Q1* (IIa)
-Q2=-Q2′-A-(C═O)-D-Q2″-A′-(C═O)-D′-Q2* (IIb)
where A is —NH—, —O— or —S— and D is —NH— in each case independently in formulae IIIa and IIIb,
where A′ is —NH— and D′ is —NH—, —O— or —S— in each case independently in formulae IIa and IIb,
where each radical Q1 and Q2 of the formula IIa or IIb has in each case independently at least two bivalent groups selected from carbamate and urea group, or
b) reacting a polysiloxane of the formula VI with a UV/Vis chromophore isocyanate selected from the formulae IXa, IXb, IXc, IXd or mixtures thereof
HA-Q2′-B-Q1′-AH (VI)
Q2*-O(CO)NH-″2Q-NCO (IXa)
Q1*-O(CO)NH-″1Q-NCO (IXb)
Q2*-O(CO)NH-Q2″-NCO (IXc)
Q1*-O(CO)NH-Q1″-NCO (IXd)
where A is —NH—, —O— or —S— and D is —NH— in each case independently in formulae VII, I and VI,
where Q1′ and Q2′ in each case independently comprises a bivalent hydrocarbon radical with 6 to 200 carbon atoms optionally comprising at least
one heteroatom O, N or S, a bivalent radical comprising aryl, arylalkyl groups or a bivalent radical comprising aryl, arylalkyl groups optionally comprising at least one heteroatom O, N or S or alkyl-, aryl- or alkyl- and aryl-group containing polyether radicals, in each case independently in formulae
VII, I und VI,
where Q1″ and Q2″ in each case independently comprises a bivalent linear, branched and/or cyclic alkyl radical with 4 to 200 carbon atoms, or a bivalent radical comprising an aryl and/or arylalkyl radical with 6 to 200 carbon atoms, in each case independently in formulae VII, I, IXa, IXb, IXc and IXd
where —O-Q1* or —O-Q2* in each case independently as -Q1* and -Q2* a UV/Vis chromophore as radical.

18. The process according to claim 17, wherein
(i) the hydroxy- or amino-functional UV/Vis chromophore is a UV chromophore comprising hydroxy-coumarin, amino-coumarin or a derivative of coumarin,
(ii) the UV/Vis chromophore isocyanate is a UV chromophore selected from coumarin isocyanates from the formulae IXa, IXb, IXc and IXd, where —O-Q1* and —O-Q2* in each case independently as radical -Q1* and/or -Q2* are coumarin or coumarin derivative, and/or
(iii) in formula IIa, IIb, IXa, IXb, IXc and/or IXd and I —O-Q1* and —O-Q2*, in each case independently comprise radicals from the reaction of hydroxycoumarin or hydroxycoumarin derivative with a diisocyanate.

19. The process according to claim 17, comprising reacting
a polysiloxane of the formula VI
HA-Q2′-B-Q1′-AH (VI)
where A is selected from −O, —NH or AH is selected from —OH,
—NH₂ or —SH with -Q2′- and -Q1′-,
with a diisocyanate to give a polysiloxane diisocyanate of the formula VII,
OCN-Q2″-D-(O═C)-A-Q2′-B-Q1′-A-(C═O)-D-Q1″-NCO (VII)
where -Q2″- and/or -Q1″- are independently selected from a bivalent, linear, branched and/or cyclic alkyl radical with 4 to 200 carbon atoms, or a bivalent radical comprising an aryl and/or arylalkyl radical with 6 to 200
carbon atoms, where the molar ratio of HA groups in the polysiloxane to isocyanate groups in formula VII is at least 1:1.

20. The process according to claim 17, which
comprises reacting
(i) a) a polysiloxane group-containing linear and/or branched polymer block B, in particular of the formulae IIIa and/or IIIb, with at least two terminal Si—H groups or at least one terminal Si—H group and at least one lateral Si—H group, or b) a polysiloxane group of the formula XI where at least one R¹⁷ is hydrogen, with
(ii) an olefinic compound comprising alkylene and optionally comprising at least one heteroatom N or O,
where the olefinic compound in each case independently has an allyl or vinyl group and corresponds to the formulae VIIIa and/or VIIIb
Q1′-AH (VIIIa)
Q2′-AH (VIIIb)
in the presence of
(iii) a catalyst to give a polysiloxane of the formula VI
HA-Q2′-B-Q1′-AH (VI)
where in each case independently in formulae VIIIa, VIIIb and VI with AH independently selected from —OH and —NH₂, and with -Q2′- and -Q1′- in each case independently in formulae VIIIa, VIIIb and VI comprising a bivalent hydrocarbon radical with 6 to 200 carbon atoms optionally comprising at least one heteroatom O or N, a bivalent radical comprising aryl, arylalkyl groups optionally comprising at least one heteroatom O or N or olefinic polyether.

21. The process according to claim 17, which comprises
reacting a polysiloxane diisocyanate of the formula VII,
OCN-Q2″-D-(O═C)-A-Q2′-B-Q1′-A-(C═O)-D-Q1″-NCO (VII)
where B is a linear and/or branched polysiloxane polymer block B, with -Q2′- and -Q1′- in each case independently comprising a bivalent hydrocarbon radical with 6 to 200 carbon atoms optionally comprising at least one heteroatom O, N or S, a bivalent radical comprising aryl, arylalkyl groups optionally comprising at least one heteroatom O, N or S, or polyether radicals containing alkyl, aryl or alkyl and aryl groups,
where A is in each case independently —NH—, —O— or —S— and D is —NH— in each case independently in formula VII, and with -Q2″- and/or -Q1″- independently selected from a bivalent, linear, branched and/or cyclic alkyl radical with 4 to 200 carbon atoms, or a bivalent radical comprising an aryl and/or arylalkyl radical with 6 to 200 carbon atoms,
with a hydroxy- or amino-functional UV/Vis chromophore.

22. The process according to claim 17, wherein
the chromophore is a hydroxycoumarin or hydroxycoumarin derivative.

23. The process according to claim 17, comprising
reacting a diisocyanate with a hydroxy-coumarin or hydroxy-coumarin derivative or salt thereof to give a coumarin isocyanate.

24. A composition obtained by the process according to claim 17.

25. A composition comprising the siloxane polymers according to claim 14, and mixtures thereof comprising
a) siloxane polymers of the general formula XI, and mixtures thereof or
b) siloxane polymers with a central polysiloxane polymer block B selected from (i) at least one siloxane polymer of the general formula I, and mixtures thereof,
(ii) at least one siloxane polymer of the general formula Ia, and mixtures thereof or
(iii) at least one siloxane polymer of the general formula Ib, and mixtures thereof.

26. An intermediate for the preparation of siloxane polymers of the formula I according to claim 1, selected from coumarin isocyanates, or salts or mixtures thereof, of formulae IXa, IXb, IXc and IXd,
Q2*-O(CO)NH-″2Q-NCO (IXa)
and/or
Q1*-O(CO)NH-″1Q-NCO (IXb)
Q2*-O(CO)NH-Q2″-NCO (IXc)
and/or
Q1*-O(CO)NH-Q1″-NCO (IXd)
where Q2* and Q1* are in each case independently coumarin or a coumarin derivative, preferably coumarin isocyanates of the formulae IXa*, IXb*, IXc*, IXd* or salts thereof,

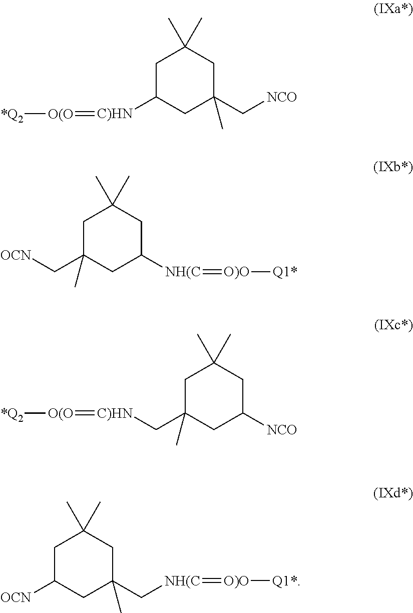

27. A formulation comprising at least one siloxane polymer according to claim 1.

28. A method of utilizing the siloxane polymers according to claim 1 as additives in cosmetic formulations, as additives in pharmaceutical formulations, in paints, pastes, as foam stabilizers or foam additives for polyurethane foams, as hand improvers or impregnating agents during the during the production of fibres, textiles, in cosmetic formulations for the treatment, post-treatment and protection of keratin fibres, and skin and skin appendages, as additives in detergents, fabric softener formulations, in cosmetic formulations including creams, rinses, hair washing compositions, washing compositions, setting agents, care rinses, care pastes, sprays, hairsprays, for improving the combability of keratin or textile fibres of natural or synthetic origin.
